SEQUENCE LISTING (20 sequences)

SEQ ID NO: 1 - RNA, 48 nt, Artificial Sequence
  Synthetic Polynucleotide
ccgucaaagcuucacaguguuucaagagaacacugugaagcuuugacg

SEQ ID NO: 2 - RNA, 19 nt, Artificial Sequence
  Synthetic Polynucleotide
cgucaaagcuucacagugu

SEQ ID NO: 3 - RNA, 19 nt, Artificial Sequence
  Synthetic Polynucleotide
acacugugaagcuuugacg

SEQ ID NO: 4 - RNA, 48 nt, Artificial Sequence
  Synthetic Polynucleotide
aagaacucgccauauagcagccucgaggcugcuauauggcgaguucuu

SEQ ID NO: 5 - RNA, 21 nt, Artificial Sequence
  Synthetic Polynucleotide
aagaacucgccauauagcagc

SEQ ID NO: 6 - RNA, 21 nt, Artificial Sequence
  Synthetic Polynucleotide
gcugcuauauggcgaguucuu

SEQ ID NO: 7 - RNA, 9 nt, Artificial Sequence
  Synthetic Polynucleotide
uucaagaga

SEQ ID NO: 8 - RNA, 6 nt, Artificial Sequence
  Synthetic Polynucleotide
cucgag

SEQ ID NO: 9 - DNA, 1758 bp, Homo sapiens
  misc_feature (213)..(213): n is a, c, g, or t
  misc_feature (1410)..(1410): n is a, c, g, or t
atgaccatcacttacacaagccaagtggctaatgcccgcttaggctccttctcccgcctgctgctgtgctggcggggcagcatctacaagctgctatatggcgagttcttaatcttcctgctctgctactacatcatccgctttatttataggctggccctcacggaagaacaacagctgatgtttgagaaactgactctgtattgcgacagntacatccagctcatccccatttccttcgtgctgggcttctacgtgacgctggtcgtgacccgctggtggaaccagtacgagaacctgccgtggcccgaccgcctcatgagcctggtgtcgggcttcgtcgaaggcaaggacgagcaaggccggctgctgcggcgcacgctcatccgctacgccaacctgggcaacgtgctcatcctgcgcagcgtcagcaccgcagtctacaagcgcttccccagcgcccagcacctggtgcaagcaggctttatgactccggcagaacacaagcagttggagaaactgagcctaccacacaacatgttctgggtgccctgggtgtggtttgccaacctgtcaatgaaggcgtggcttggaggtcgaatccgggaccctatcctgctccagagcctgctgaacgagatgaacaccttgcgtactcagtgtggacacctgtatgcctacgactggattagtatcccactggtgtatacacaggtggtgactgtggcggtgtacagcttcttcctgacttgtctagttgggcggcagtttctgaacccagccaaggcctaccctggccatgagctggacctcgttgtgcccgtcttcacgttcctgcagttcttcttctatgttggctggctgaaggtggcagagcagctcatcaacccctttggagaggatgatgatgattttgagaccaactggattgtcgacaggaatttgcaggtgtccctgttggctgtggatgagatgcaccaggacctgcctcggatggagccggacatgtactggaataagcccgagccacagcccccctacacagctgcttccgcccagttccgtcgagcctcctttatgggctccaccttcaacatcagcctgaacaaagaggagatggagttccagcccaatcaggaggacgaggaggatgctcacgctggcatcattggccgcttcctaggcctgcagtcccatgatcaccatcctcccagggcaaactcaaggaccaaactactgtggcccaagagggaatcccttctccacgagggcctgcccaaaaaccacaaggcagccaaacagaacgttaggggccaggaagacaacaaggcctggaagcttaaggctgtggacgccttcaagtctgccccactgtatcagaggccaggctactacagtgccccacagacncccctcagccccactcccatgttcttccccctagaaccatcagcgccgtcaaagcttcacagtgtcacaggcatagacaccaaagacaaaagcttaaagactgtgagttctggggccaagaaaagttttgaattgctctcagagagcgatggggccttgatggagcacccagaagtatctcaagtgaggaggaaaactgtggagtttaacctgacggatatgccagagatccccgaaaatcacctcaaagaacctttggaacaatcaccaaccaacatacacactacactcaaagatcacatggatccttattgggccttggaaaacagggatgaagcacattcctaa

SEQ ID NO: 10 - DNA, 20 bp, Artificial Sequence
  Synthetic Polynucleotide
ctactgtacggagaatttct

SEQ ID NO: 11 - DNA, 19 bp, Artificial Sequence
  Synthetic Polynucleotide
ccagcaagctgcacagcgt

SEQ ID NO: 12 - DNA, 623 bp, Artificial Sequence
  Synthetic Polynucleotide
aattctgtcattttactagggtgatgaaattcccaagcaacaccatccttttcagataagggcactgaggctgagagaggagctgaaacctacccggcgtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggaccaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagacc

SEQ ID NO: 13 - DNA, 114 bp, Artificial Sequence
  Synthetic Polynucleotide
taaaacgacggccagtgaattcatatttgcatgtcgctatgtgttctgggaaatcaccataaacgtgaaatgtctttggatttgggaatcttataagttctgtatgagaccact

SEQ ID NO: 14 - DNA, 241 bp, Artificial Sequence
  Synthetic Polynucleotide
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 15 - DNA, 24 bp, Homo sapiens
aagctgctatatggcgagttctta

SEQ ID NO: 16 - DNA, 21 bp, Artificial Sequence
  Synthetic Polynucleotide
cgacgatataccgctcaagaa

SEQ ID NO: 17 - DNA, 24 bp, Artificial Sequence
  Synthetic Polynucleotide
aagctgctgtacggcgagttcctg

SEQ ID NO: 18 - DNA, 48 bp, Artificial Sequence
  Synthetic Polynucleotide
ccgtcaaagcttcacagtgtttcaagagaacactgtgaagctttgacg

SEQ ID NO: 19 - DNA, 48 bp, Artificial Sequence
  Synthetic Polynucleotide
gctgctatatggcgagttcttctcgagaagaactcgccatatagcagc

SEQ ID NO: 20 - DNA, 174 bp, Artificial Sequence
  Synthetic Polynucleotide
taaaacgacggccagtgaattcatatttgcatgtcgctatgtgttctgggaaatcaccataaacgtgaaatgtctttggatttgggaatcttataagttctgtatgagaccactcggatcccgtcaaagcttcacagtgtttcaagagaacactgtgaagctttgacgtttttt

(19) United States
(12) Patent Application Publication
(10) Pub. No.: US 2022/0033826 A1
(43) Pub. Date: Feb. 3, 2022

(54) ADENO-ASSOCIATED VIRAL VECTORS FOR THE TREATMENT OF BEST DISEASE

(71) Applicant: University of Florida Research Foundation, Incorporated, Gainesville, FL (US)

(72) Inventors: William W. Hauswirth, Gainesville, FL (US); Alfred S. Lewin, Gainesville, FL (US); Cristhian J. Ildefonso, Gainesville, FL (US); Brianna M. Young, Ocklawaha, FL (US)

(73) Assignee: University of Florida Research Foundation, Incorporated, Gainesville, FL (US)

(21) Appl. No.: 17/272,203

(22) PCT Filed: Aug. 30, 2019
(86) PCT No.: PCT/US2019/049163

§ 371 Date: Feb. 26, 2021

Related U.S. Application Data
(60) Provisional application No. 62/754,530, filed on Nov. 1, 2018.
(60) Provisional application No. 62/749,622, filed on Oct. 23, 2018.
(60) Provisional application No. 62/726,184, filed on Aug. 31, 2018.

(51) Int. Cl. C12N 15/113; C12N 15/86; C12N 7/00; C07K 14/705; A61K 48/00

(52) U.S. Cl. CPC C12N 15/1138; C12N 15/86; C12N 7/00; C07K 14/705; C12N 2750/14143; C12N 2310/531; C12N 2800/22; C12N 2310/14; C12N 2320/30; A61K 48/005

(57) ABSTRACT

Aspects of the disclosure relate to methods and compositions useful for treating bestrophinopathies, such as Best Disease.

### CROSS-REFERENCE TO RELATED APPLICATIONS

This application is a national stage filing under 35 U.S.C. § 371 of International Application, PCT/US2019/049163 filed Aug. 30, 2019, which claims the benefit of the filing dates of U.S. Provisional Application No. 62/726,184 filed Aug. 31, 2018, U.S. Provisional Application No. 62/749,622 filed Oct. 23, 2018, and U.S. Provisional Application No. 62/754,530, filed Nov. 1, 2018, the entire contents of each of which are incorporated by reference.

### GOVERNMENT SUPPORT

The invention was made with government support under Grant No. EY021721 awarded by the National Institutes of Health. The government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Sep. 8, 2021, is named U119670061US03-SUBSEQ-EPG and is 8,056 bytes in size.

### BACKGROUND

Mutations in the BEST1 gene (also called VMD2) cause several forms of retinal degeneration including Best vitelliform macular dystrophy, also known as Best Disease. (Best Disease may also be referred to as Best macular dystrophy, vitelline dystrophy, and vitelliform macular dystrophy.) Bestrophinopathies are caused by more than 200 different mutations in the human BEST1 gene that encodes a protein (bestrophin, or BEST1) that functions as a calcium-dependent chloride channel associated with basolateral membrane of the retinal pigment epithelium. In bestrophinopathies, defective fluid transport across the RPE damages the interaction between the RPE and photoreceptor cells. This damage leads to detachment of the retina from its supporting layer and accumulation of oxidized proteolipid (lipofuscin) within the RPE and subretinal space. Eventually, photoreceptors die, primarily in the macular region, which is responsible for central vision. In humans, BEST1 mutations are usually autosomal dominant, meaning that one defective copy leads to disease regardless of the presence of a normal (wild type) gene inherited from the other parent. However, autosomal recessive bestrophinopathies (ARBs) have also been reported.

Best Disease, a rare disease, is a slowly progressive macular dystrophy with onset generally in childhood and sometimes in later teenage years. Affected individuals initially have normal vision followed by decreased central visual acuity and metamorphopsia. Individuals retain normal peripheral vision and dark adaptation. Individuals develop a mass on the macula that resembles an egg yolk. This mass eventually breaks up and spreads throughout the macula, leading to a reduction in central vision. Best Disease may be diagnosed based on family history or ophthalmologic examination, e.g., fundus appearance or electrooculogram (EOG).

Inherited retinal degenerations (IRDs) encompass a large group of blinding conditions that are molecularly heterogeneous and pathophysiologically distinct. The genetic defect often acts primarily on rod or cone photoreceptors (PRs), or both, and the specific defect may involve phototransduction, ciliary transport, morphogenesis, neurotransmission, or others. Less common are primary defects involving the retinal pigment epithelium (RPE), although they have received increased attention due to high-profile clinical trials.

The most common IRD due to a primary RPE defect is caused by mutations in BEST1, encoding a transmembrane protein associated with the basolateral portion of the RPE. BEST1 (bestrophin) is a multifunctional channel protein responsible for mediating transepithelial ion transport, regulation of intracellular calcium signaling and RPE cell volume, and modulation of the homeostatic milieu in the subretinal space. In eukaryotic cells, BEST1 forms a stable homopentamer with four transmembrane helices, cytosolic N and C termini, and a continuous central pore sensitive to calcium-dependent control of chloride permeation.

In humans, BEST1 mutations result in a wide spectrum of IRDs collectively grouped as bestrophinopathies that often involve pathognomonic macular lesions. Retinal regions away from the lesions tend to appear grossly normal, despite the existence of a retina-wide electrophysiological defect in the EOG, which reflects an abnormality in the standing potential of the eye. Naturally-occurring biallelic mutations in the canine BEST1 gene (cBEST1) cause canine IRD with distinct phenotypic similarities to both the dominant and recessive forms of human bestrophinopathies, including the salient predilection of subretinal lesions to the canine fovea-like area.

Proper anatomical apposition and a sustained interaction between RPE apical microvilli (MV) and PR outer segments (OSs) are considered crucial for normal vision. Both the ionic composition and volume regulation of the subretinal space are essential for maintaining the accurate molecular proximity of this complex and homeostasis of the RPE-PR interface. In vitro and ex vivo studies have long shown that genetic mutations, metabolic perturbations, as well as light stimuli alter the ionic composition of the subretinal space and physiological responses of the RPE and/or PRs. More recently, in vivo studies of outer retinal microanatomy in health and disease and its response to light have become increasingly informative with modern retinal imaging modalities.

Mutations in the BEST1 gene cause detachment of the retina and degeneration of photoreceptor (PR) cells due to a primary channelopathy in the neighboring retinal pigment epithelium (RPE) cells. The pathophysiology of the interaction between RPE and PR cells preceding the formation of retinal detachment remains not well-understood.

### SUMMARY OF THE INVENTION

Aspects of the disclosure relate to compositions for treating bestrophinopathies (e.g., Best vitelliform macular dystrophy) in a subject (e.g., in a human). Aspects of the disclosure are designed to suppress the expression of endogenous BEST1 mRNA (e.g., both the mutated and the normal copy). In some embodiments, the expression is suppressed using RNA interference. In some embodiments, the endogenous BEST1 mRNA is simultaneously replaced with normal BEST1 mRNA to produce only normal protein. In some embodiments, adeno-associated virus (AAV) is used to deliver an intronless copy of the BEST1 gene plus a gene for a small hairpin RNA (shRNA) that leads to the production of a small interfering RNA (siRNA).

In some embodiments, one or both alleles of the BEST1 gene of a subject (e.g., a human) are silenced by administering a short hairpin RNA (shRNA) molecule to a subject (e.g., to a subject having Best Disease, for example to a human having Best Disease). In some embodiments, a replacement BEST1 coding sequence also is administered to the subject to provide a functional bestrophin protein, e.g., to restore photoreceptor function to the subject. In some embodiments, the replacement BEST1 coding sequence has one or more nucleotide substitutions relative to the endogenous gene allele(s) that render the replacement gene resistant to the effects of the interfering RNA. In some embodiments, the replacement BEST1 coding sequence is a human BEST1 coding sequence (e.g., a wild-type human BEST1 coding sequence) that includes one or more (e.g., 1, 2, 3, 4, 5, or more) substitutions to render the gene resistant to degradation mediated by the shRNA. In some embodiments, the replacement BEST1 coding sequence includes one or more silent mutations (base changes in the third position of codons) in the target site to render the gene “de-targeted” to degradation mediated by the shRNA.

In some aspects, the disclosure provides a short hairpin RNA (shRNA) comprising a sense strand comprising the nucleotide sequence CGUCAAAGCUUCACAGUGU (SEQ ID NO: 2), an antisense strand comprising the nucleotide sequence ACACUGUGAAGCUUUGACG (SEQ ID NO: 3), and a loop. In some embodiments, the loop comprises the nucleotide sequence UUCAAGAGA (SEQ ID NO: 7).

In some aspects, the disclosure provides a short hairpin RNA (shRNA) comprising a sense strand comprising the nucleotide sequence GCUGCUAUAUGGCGAGUUCUU (SEQ ID NO: 6), an antisense strand comprising the nucleotide sequence AAGAACUCGCCAUAUAGCAGC (SEQ ID NO: 5), and a loop. In some embodiments, the loop comprises the nucleotide sequence CUCGAG (SEQ ID NO: 8).

In some embodiments, the disclosure provides a short hairpin RNA (shRNA) that comprises an antisense strand comprising the nucleotide sequence ACACUGUGAAGCUUUGACG (SEQ ID NO: 3).

In some aspects, the disclosure provides a vector comprising a genetic sequence encoding the shRNAs described in the preceding paragraphs.

In some aspects, the disclosure provides a vector that further comprises a recombinant functional (e.g., wild-type) BEST1 coding sequence that does not contain a sequence targeted by the shRNA. In some aspects, the vector further comprises a recombinant functional BEST1 coding sequence that is codon-optimized for expression in a human cell.

In some aspects, the disclosure provides a vector that comprises a recombinant BEST1 coding sequence that comprises a nucleotide sequence that is at least 90% identical to the nucleotide sequence of SEQ ID NO: 9. In some aspects, the disclosure provides a vector that comprises a recombinant BEST1 coding sequence that comprises a nucleotide sequence that is at least 90% identical to the nucleotide sequence of SEQ ID NO: 10.

In some aspects, the disclosure provides a vector that is a plasmid or a viral vector. In some aspects, the viral vector is a recombinant adeno-associated viral (rAAV) vector. In some aspects, the rAAV vector is self-complementary.

In some aspects, the disclosure provides a rAAV viral particle that is an AAV serotype 2 viral particle.

In some aspects, the disclosure provides a composition comprising a vector or rAAV particle and a pharmaceutically acceptable carrier.

In some aspects, the disclosure provides a method of modulating BEST1 expression in a subject, the method comprising administering to the subject, such as a human subject, a composition comprising a vector or rAAV particle and a pharmaceutically acceptable carrier. In some aspects, the disclosure provides a method of treating bestrophinopathies (e.g., Best Disease and ARB) in a subject, the method comprising administering a composition.

In some embodiments, a vector encoding a functional BEST1 sequence is provided to supplement or correct (e.g., at least partially) cellular BEST1 function without knocking down endogenous BEST1 gene expression. In some embodiments, the BEST1 sequence is codon-optimized.

In some embodiments, a vector encoding a functional BEST1 sequence is provided to supplement or correct (e.g., at least partially) cellular BEST1 function and an shRNA sequence is provided to knock down endogenous BEST1 gene expression. In some embodiments, endogenous Best1 expression is knocked down using shRNA. In some embodiments, the BEST1 sequence is codon-optimized. In some embodiments, the BEST1 sequence is modified to be resistant to the shRNA. In some embodiments, the BEST1 and shRNA sequences are encoded on the same AAV vector.

In some aspects, the disclosure provides a composition for use in treating Best Disease and a composition for use in the manufacture of a medicament to treat Best Disease. In some aspects, the disclosure provides a composition comprising a vector or rAAV particle, wherein the vector encodes a functional BEST1 sequence, for use in treating ARB, and a composition for use in the manufacture of a medicament to treat ARB.

These and other aspects are described in the following drawings, examples, and claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1D show retina-wide pathology of RPE apical microvillar projections associated with BEST1 mutations in canines. FIGS. 1A and 1B show confocal images illustrating the molecular pathology of cBest (R25*/R25*; 89 wk) (FIG. 1B) compared with wild-type (FIG. 1A) (42 wk). Retinal cryosections were immunolabeled with anti-EZRIN and human cone arrestin and combined with peanut agglutinin lectin and DAPI labels. FIG. 1C shows representative photomicrographs of 6-wk-old canine wild-type and cBest-mutant (R25*/P463fs) retinas immunolabeled with anti-BEST1 and anti-SLC16A1. White arrowheads point to a subset of cone-MV. FIG. 1D shows quantification of cone-MV numbers across the retina between cBest-mutant and age-matched control eyes. The y axis represents the average number of cone-MV per square millimeter for each color-coded retinal region examined. Abbreviations: H&E, he-matoxylin & eosin staining; PRL, photoreceptor IS/OS layer; i, inferior; N, nasal; S, superior; T, temporal.

FIGS. 2A-2F show light-mediated changes in the outer retinal structure in wild-type and cBest (R25*/P463fs) mutants. FIG. 2A shows cross-sectional imaging along the horizontal meridian through the central area centralis (fovea-like area) in a 15-wk-old normal (WT) dog and an 11-wk-old cBest (R25*/P463fs) with less and more light adaptation (LA). Thin white arrows indicate the superotemporal location of the OCT. FIG. 2B shows longitudinal reflectivity profiles (LRPs) (average of 85 single LRPs) at 3° nasal from the fovea-like area (T, temporal retina) and nasal edge of the optic nerve head (N, nasal retina) in WT dogs (12 eyes, age 15 to 17 wk) compared with cBest-treated dogs (6 eyes, age 11 wk) with less and more LA. Arrowheads indicate IS/OS and RPE/T peaks; single and double arrows mark the additional hyporeflective layer in cBest. FIG. 2C shows the distance between IS/OS and RPE/T peaks in WT and cBest eyes under two LA conditions. Symbols with error bars represent mean (±2 SD) distance for each group of eyes at both locations. FIG. 2D is a schematic description of the dark and light adaptation protocol. Animals were dark-adapted (D/A) overnight and OCT imaging was performed. Then, five increasing light exposures (L1 through L5) were used. FIG. 2D also shows magnified views of the OCT scans in cBest with overlapping LRPs after overnight dark adaptation (left) and after the maximum light exposure (right). FIG. 2E shows in a different subset of cBest eyes (n=3; colored traces), results of using an abbreviated protocol involving only L4 and L5 exposures. FIG. 2F shows spatial topography of IS/OS-to-RPE/T distance in mean WT compared with two representative cBest eyes [panels; EM356-OS: 297-wk-old cmr1/cmr3 (R25*/P463fs); LH30-OD: 12-wk-old cmr3 (P463fs/P463fs)].

FIGS. 3A-3D show BEST1 gene augmentation therapy results in sustained reversal of foveomacular lesions and restoration of RPE-PR interface structure in cBest mutants. FIG. 3A shows the natural history of the central subretinal detachment documented by in vivo imaging in the right eye of cBest dog (EM356-OD; R25*/P463fs) at three time points. The insets show auto-fluorescence and OCT images. FIG. 3B shows fundus images taken before (at 52 wk of age) and after subretinal injection with AAV2-cBEST1 (1.5×10¹⁰ vg/mL) was performed in the eye shown in FIG. 3A. The subretinal bleb area is denoted by the dashed circle. Images acquired at 43 and 245 wk post-injection document sustained reversal of the central lesion and fully reattached retina within the treated area. Middle and right insets show autofluorescence and OCT images. FIGS. 3C and 3D show the restoration of RPE-photoreceptor interface structure post AAV-hBEST1 treatment in the cBest (R25*/R25*) model in comparison with control. Bleb boundaries are marked by dashed circles; the locations of corresponding OCT scans cut through the subretinal lesions before injection or through the matching locations mapped post-injection are marked by horizontal lines; retinotomy sites are indicated by arrowheads.

FIGS. 4A-4F show reversal of microdetachments across retinal regions after subretinal gene therapy in cBest-mutant dogs [owl (R25*/R25*), cmr1/cmr3 (R25*/P463fs), or cmr3 (P463fs/P463fs)] subretinally injected with BSS or AAV-hBEST1. FIG. 4A shows maps of IS/OS-RPE/T distance topography in cBest-mutant dogs [owl (R25*/R25*), cmr1/cmr3 (R25*/P463fs), or cmr3 (P463fs/P463fs)] subretinally injected with BSS or AAV-hBEST1. Treatment boundaries are based on fundus photographs of the bleb taken at the time of the injection (dotted lines) and, if visible, demarcations apparent at the time of imaging (dashed lines). All eyes are shown as equivalent right eyes with optic nerve and major blood vessels (black), tapetum boundary (white), and fovea-like region (white ellipse) overlaid for ease of comparison. FIG. 4B shows the IS/OS-RPE/T distance difference from WT at the superior and inferior retinal locations in cBest eyes within the treated bleb (Tx; filled symbols) and untreated outside bleb (Ctrl; open symbols) regions. Dashed lines delimit the 95th percentile of normal variability. Topographies of the IS/OS-RPE/T distance are shown pre- (Left) and posttreatment (Right). FIGS. 4C and 4E depict grayscale maps of the difference between each cBest eye and mean WT control. White represents gross retinal detachments. FIGS. 4D and 4F show measurements of the colocalized difference of IS/OS-RPE/T distance between WT and cBest pre- (PreTx) and post treatment (Tx) for the eyes shown in FIGS. 4C and 4E, respectively.

FIGS. 5A-5G show retinotopic phenotype in two human subjects with ARB. FIG. 5A shows RPE health across the retinas of two ARB patients, P1 and P2, imaged with short-wavelength reduced-illuminance autofluorescence imaging (SW-RAFI), taking advantage of the natural RPE fluorophore lipofuscin. White arrows depict the location of the perimetric profiles and OCT scans; rectangles show the regions of interest shown in other panels; and black arrowheads demarcate disease-to-health transition in the nasal midperipheral retina. FIG. 5B shows perimetric light sensitivity of rods in dark-adapted (upper) and cones in light-adapted (lower) eyes measured across the horizontal meridian. Grey regions represent normal sensitivity except for the physiological blindspot corresponding to the optic nerve (ONH). FIG. 5C shows retinal cross-section with OCT along the horizontal meridian crossing the fovea. FIGS. 5D and 5E show detail of outer retinal lamination in patients compared with normal at the two regions of interest at the parapapillary retina (FIG. 5D) and midperipheral nasal retina (FIG. 5E). Color indicates interface near COS tips and interface near ROS tips and RPE apical processes, and brick indicates interface near the RPE and Bruch's membrane. FIGS. 5F and 5G show dark-adaptation kinetics measured in P1 at the parapapillary locus (FIG. 5F) and in P2 at the nasal midperipheral locus (FIG. 5G). Time 0 refers to the end of adaptation light.

FIGS. 6A-6D show RPE-PR interdigitation zone in canine models of CNGB3-associated achromatopsia (ACHM3). FIGS. 6A and 6B show representative fluorescence microscopy images of 6-wk-old CNGB3-D262N-mutant (FIG. 6A) and CNGB3-null (FIG. 6B; CNGB3^{-/-}) retinas demonstrating normal expression of BEST1 limited to the basolateral plasma membrane of RPE cells, and SLC16A1, a marker labeling RPE apical processes. Arrows point to a subset of cone-associated RPE apical microvilli (c-MV). FIGS. 6A and 6B also show anti-CNGB3 and anti-EZRIN colabeling, with an age-matched wild-type retina shown for reference. FIGS. 6C and 6D show immunohistochemical evaluation of the RPE-PR interface in CNGB3-mutant retinas from 85-wk-old (FIG. 6C) and 57-wk-old (FIG. 6D) affected dogs. RPE apical aspect and its microvillar extensions were immunolabeled with EZRIN, and a subset of c-MV is denoted by arrows. Abbreviations: ACHM3, achromatopsia type 3; cCNGB3, canine CNGB3 gene; c-MV, cone-associated RPE apical microvilli; CNGB3, cyclic nucleotide-gated channel beta 3 protein; hCAR, human cone arrestin; SLC16A1, solute carrier family 16 member 1.

FIG. 7 shows recovery of light-mediated microdetachments. Two cBest-affected (R25*/P463fs) eyes [ages 43 (Right) and 52 (Left) wk] were dark-adapted overnight and imaged similar to results shown in FIG. 2A.

FIGS. 8A-8B show hyperthick ONL at retinal regions with microdetachment, and its correction with gene therapy, in cBest eyes. FIG. 8A shows that uninjected cBest eyes (shown in FIGS. 2A-2F and 4A-4F as IS/OS-RPE/T thickness maps) demonstrate hyperthick ONL corresponding to large regions of retinal microdetachment, and localized thinning of the ONL above gross lesions and near the fovea-like region in some eyes. FIG. 8B shows that treated cBest eyes (shown in FIGS. 4A-4F as IS/OS-RPE/T thickness maps) demonstrate normal ONL thickness in the AAV-treated regions surrounded by hyperthick, normal, or thinned ONL within untreated regions. OD, right eye; OS, left eye.

FIGS. 9A-9F show evolution of a focal macular lesion in a cBest-affected (R25*/P463fs) dog (EM356-OS). FIG. 9A shows that the discrete separation of photoreceptor layer from the underlying RPE progressed to form a larger subretinal macrodetachment (vitelliform lesion) evident en face and FIG. 9B shows the corresponding OCT scan at 23 wk of age. FIG. 9C shows the first signs of hyper-autofluorescent material accumulating within the subretinal lesion were observed 8 wk later (31 wk; early pseudohypopyon lesion). FIG. 9D shows that at 66 wk of age, a typical pseudohypopyon appearance is documented, followed by vitelliruptive-like lesions at 172 and 297 wk of age with dispersion of the autofluorescent material (Insets, close-ups). FIGS. 9E and 9F show that significant thinning of the ONL is apparent by OCT scan. Darkened lines demarcate the position of the corresponding SD-OCT scans.

FIG. 10 shows Retinal preservation after AAV-hBEST1 treatment in three cBest models [cmr1 (R25*/R25*), cmr1/cmr3 (R25*/P463fs), and cmr3 (P463fs/P463fs)] in comparison with the wild-type control and cBest untreated eyes.

FIGS. 11A-11D show dose-response effects of BEST1 transgene expression on RPE cytoskeleton rescue in a cBest (R25*/P463fs) retina. FIG. 11A shows a cross-sectional overview from the surgical bleb area (left), through the adjacent penumbral region (middle), and toward the contiguous extent outside of the injection zone (right). FIG. 11B shows the remarkable extension of RPE apical projections within the treated region with augmented BEST1; FIG. 11C shows the presence of vestigial microvilli and rod-MV in the bleb penumbra associated with patchy distribution of BEST1 (weak signals within individual RPE cells) and RPE-PR microdetachment; FIG. 11D shows the formation of subretinal lesions in the absence of both BEST1 expression and RPE apical processes outside of the treatment zone.

FIGS. 12A-12B shows interocular symmetry of rod and cone function ARB patients P1 (FIG. 12A) and P2 (FIG. 12B). Rod (RSL) and cone sensitivity loss (CSL) maps of both eyes of two patients with ARB.

FIG. 13 shows a map of 6262-bp plasmid, pTR-VMD2-hBest, human Bestrophin.

FIG. 14 shows a map of 6222-bp plasmid, pTR-VMD2-cBest, canine Bestrophin.

FIG. 15 shows a map of 6209-bp plasmid, pTR-SB-VMD2-HBest1-shRNA05, which contains resistant Best1.

FIG. 16 shows a map of 6145-bp plasmid, pTR-SB-VMD2-DTBest1-shRNA744, which contains de-targeted Best1.

FIG. 17 shows that the VMD2 promoter works well in cell culture. HEK293T cells were transfected with plasmids expressing GFP or Best1 using the Chicken beta actin promoter (CBA) or the VMD2 promoter. Protein lysates were separated on polyacrylamide gels and expression of bestrophin (Best1) was detected by Western Blot and normalized to the expression of beta-tubulin to show even loading of the gel.

FIGS. 18A-18B show that Best1 specific-siRNA is functional. The band intensities shown in the Western blot (FIG. 18A) and quantified in a bar graph (FIG. 18B) indicate that the transfection of HEK293T stably expressing BEST1 led to a 75% reduction in Bestrophin (Best1) protein.

FIGS. 19A-19B show that Best1 shRNA is active: HEK293T-BEST1 cells were transfected with 4 μg of the indicated plasmid.

FIG. 20 shows the detargeting of Best1. Silent mutations (base changes in the third position of codons) were used to remove an siRNA target site from Best1 mRNA. The example disclosed is for shRNA744. SEQ ID NOs: 15-17 correspond to the sequences from top to bottom: wild-type BEST1 target site; the (complementary) shRNA744 target site, and de-targeted DTBEST1 siRNA target site.

### DETAILED DESCRIPTION

Aspects of the application provide methods and compositions that are useful for treating Best Disease in a subject (e.g., in a human subject having Best Disease).

In some embodiments, the disclosure provides methods and compositions for delivering a functional bestrophin protein to subjects having one or more mutant BEST1 genes. In some embodiments, a recombinant BEST1 gene (e.g., a coding sequence, for example a cDNA or open reading frame) is provided on a viral vector (e.g., an rAAV vector). In some embodiments, expression of one or both alleles of the endogenous BEST1 gene are also knocked down. For example, in some embodiments an siRNA (e.g., an shRNA) is delivered to a subject along with a recombinant BEST1 gene. In some embodiments, a viral vector (e.g., an rAAV vector) encodes both a recombinant BEST1 gene and one or more siRNAs that target the endogenous BEST1 gene. In some embodiments, the recombinant BEST1 gene is modified to comprise one or more nucleotide substitutions that make it resistant to targeting by the one or more siRNAs. In some embodiments, the recombinant BEST1 gene is codon optimized (e.g., for expression in a subject, for example in a human subject).

In some embodiments, the disclosure provides a short hairpin RNA (shRNA) comprising a sense strand comprising the nucleotide sequence CGUCAAAGCUUCACAGUGU (SEQ ID NO: 2), an antisense strand comprising the nucleotide sequence ACACUGUGAAGCUUUGACG (SEQ ID NO: 3), and a loop. In some embodiments, the loop comprises the nucleotide sequence UUCAAGAGA (SEQ ID NO: 7)

In other embodiments, the disclosure provides a short hairpin RNA (shRNA) comprising a sense strand comprising the nucleotide sequence GCUGCUAUAUGGCGAGUUCUU (SEQ ID NO: 6), an antisense strand comprising the nucleotide sequence AAGAACUCGCCAUAUAGCAGC (SEQ ID NO: 5), and a loop. In some embodiments, the loop comprises the nucleotide sequence CUCGAG (SEQ ID NO: 8).

In some embodiments, the disclosure provides a short hairpin RNA (shRNA) that comprises an antisense strand comprising the nucleotide sequence ACACUGUGAAGCUUUGACG (SEQ ID NO: 3).

In some embodiments, the shRNA can be delivered using a vector as an shRNA driven by a promoter (e.g., a human H1 RNA promoter). In some embodiments, this vector is a plasmid. In some embodiments, the vector is a viral vector, such as an adeno-associated virus (AAV) vector. In some embodiments, the vector is a double-stranded or self-complementary AAV vector. In some embodiments, the vector sequence encoding the shRNA comprises a BEST1 sequence.

Accordingly, in some embodiments an shRNA can be encoded on a DNA vector (e.g., a viral vector) by a nucleic acid having a sequence of

(SEQ ID NO: 18)
CCGTCAAAGCTTCACAGTGTTTCAA
GAGAACACTGTGAAGCTTTGACG,

where the loop sequence is underlined. In some embodiments, a different loop sequence is substituted for the loop sequence shown in SEQ ID NO: 7.

Also, in some embodiments, an shRNA can be encoded on a DNA vector (e.g., a viral vector) by a nucleic acid having a sequence of

(SEQ ID NO: 19)
GCTGCTATATGGCGAGTTCTTCTCG
AGAAGAACTCGCCATATAGCAGC,

where the loop sequence is underlined. In some embodiments, a different loop sequence is substituted for the loop sequence shown in SEQ ID NO: 8.

In some embodiments, the same vector comprises a coding sequence that encodes normal (e.g., wild-type) Best1 protein but is resistant to the action of the shRNA expressed by the vector.

In some embodiments, the BEST1 coding sequence comprises a sequence that is at least 90%, at least 91%, at least 92%, at least 93%, at least 94%, at least 95%, at least 96%, at least 97%, at least 98%, at least 99%, or 100% identical to SEQ ID NO: 9.

In some embodiments, the BEST1 coding sequence comprises a sequence that is at least 90%, at least 91%, at least 92%, at least 93%, at least 94%, at least 95%, at least 96%, at least 97%, at least 98%, at least 99%, or 100% identical to SEQ ID NO: 10. In some embodiments, the BEST1 coding sequence comprises a sequence that is at least 90%, at least 91%, at least 92%, at least 93%, at least 94%, at least 95%, at least 96%, at least 97%, at least 98%, at least 99%, or 100% identical to SEQ ID NO: 11.

In some embodiments, the BEST1 coding sequence comprises a sequence that is at least 90%, at least 91%, at least 92%, at least 93%, at least 94%, at least 95%, at least 96%, at least 97%, at least 98%, at least 99%, or 100% identical to SEQ ID NO: 15.

The 1757-bp wild-type BEST1 sequence is
defined as follows (SEQ ID NO: 9):
ATGACCATCACTTACACAAGCCAAGTGGCTAATGC
CCGCTTAGGCTCCTTCTCCCGCCTGCTGCTGTGCT
GGCGGGGCAGCATCTACAAGCTGCTATATGGCGAG
TTCTTAATCTTCCTGCTCTGCTACTACATCATCCG
CTTTATTTATAGGCTGGCCCTCACGGAAGAACAAC
AGCTGATGTTTGAGAAACTGACTCTGTATTGCGAC
AGNTACATCCAGCTCATCCCCATTTCCTTCGTGCT
GGGCTTCTACGTGACGCTGGTCGTGACCCGCTGGT
GGAACCAGTACGAGAACCTGCCGTGGCCCGACCGC
CTCATGAGCCTGGTGTCGGGCTTCGTCGAAGGCAA
GGACGAGCAAGGCCGGCTGCTGCGGCGCACGCTCA
TCCGCTACGCCAACCTGGGCAACGTGCTCATCCTG
CGCAGCGTCAGCACCGCAGTCTACAAGCGCTTCCC
CAGCGCCCAGCACCTGGTGCAAGCAGGCTTTATGA
CTCCGGCAGAACACAAGCAGTTGGAGAAACTGAGC
CTACCACACAACATGTTCTGGGTGCCCTGGGTGTG
GTTTGCCAACCTGTCAATGAAGGCGTGGCTTGGAG
GTCGAATCCGGGACCCTATCCTGCTCCAGAGCCTG
CTGAACGAGATGAACACCTTGCGTACTCAGTGTGG
ACACCTGTATGCCTACGACTGGATTAGTATCCCAC
TGGTGTATACACAGGTGGTGACTGTGGCGGTGTAC
AGCTTCTTCCTGACTTGTCTAGTTGGGCGGCAGTT
TCTGAACCCAGCCAAGGCCTACCCTGGCCATGAGC
TGGACCTCGTTGTGCCCGTCTTCACGTTCCTGCAG
TTCTTCTTCTATGTTGGCTGGCTGAAGGTGGCAGA
GCAGCTCATCAACCCCTTTGGAGAGGATGATGATG
ATTTTGAGACCAACTGGATTGTCGACAGGAATTTG
CAGGTGTCCCTGTTGGCTGTGGATGAGATGCACCA
GGACCTGCCTCGGATGGAGCCGGACATGTACTGGA
ATAAGCCCGAGCCACAGCCCCCCTACACAGCTGCT
TCCGCCCAGTTCCGTCGAGCCTCCTTTATGGGCTC
CACCTTCAACATCAGCCTGAACAAAGAGGAGATGG
AGTTCCAGCCCAATCAGGAGGACGAGGAGGATGCT
CACGCTGGCATCATTGGCCGCTTCCTAGGCCTGCA
GTCCCATGATCACCATCCTCCCAGGGCAAACTCAA
GGACCAAACTACTGTGGCCCAAGAGGGAATCCCTT
CTCCACGAGGGCCTGCCCAAAAACCACAAGGCAGC
CAAACAGAACGTTAGGGGCCAGGAAGACAACAAGG
CCTGGAAGCTTAAGGCTGTGGACGCCTTCAAGTCT
GCCCCACTGTATCAGAGGCCAGGCTACTACAGTGC
CCCACAGACNCCCCTCAGCCCCACTCCCATGTTCT
TCCCCCTAGAACCATCAGCGCCGTCAAAGCTTCAC
AGTGTCACAGGCATAGACACCAAAGACAAAAGCTT
AAAGACTGTGAGTTCTGGGGCCAAGAAAAGTTTTG
AATTGCTCTCAGAGAGCGATGGGGCCTTGATGGAG
CACCCAGAAGTATCTCAAGTGAGGAGGAAAACTGT
GGAGTTTAACCTGACGGATATGCCAGAGATCCCCG
AAAATCACCTCAAAGAACCTTTGGAACAATCACCA
ACCAACATACACACTACACTCAAAGATCACATGGA
TCCTTATTGGGCCTTGGAAAACAGGGATGAAGCAC
ATTCCTAA

In some embodiments, the BEST1 coding sequence comprises includes a short de-targeted sequence that corresponds a region of the wild-type BEST1 gene. An exemplary de-targeted sequence that may be used with a vector sequence encoding an shRNA744 sequence is defined as follows (SEQ ID NO: 10): CTACTGTACGGAGAATTTCT.

Other nucleotide substitutions can be made to de-target the BEST1 sequence. For example, in some embodiments, the de-targeted sequence is located in a different position on the BEST1 coding sequence and corresponds to a different region of the wild-type BEST1 gene. An exemplary de-targeted sequence that may be used with a vector sequence encoding an shRNA05 sequence and is defined as follows (SEQ ID NO: 11): CCAGCAAGCTGCACAGCGT.

In some embodiments, an shRNA (e.g., shRNA05) encoded by a nucleic acid comprising the sequence of SEQ ID NO: 1 (and/or the complement thereof) is transcribed in a host cell (e.g., in a subject, for example in a human subject) treated with the vector. In some embodiments, two or more different shRNAs (e.g., having different start sites and/or termination sites, for example differing from shRNA05 by one or two additional or fewer nucleotides) are transcribed in a host cell.

In some embodiments, the BEST1 coding sequence is driven by a promoter (e.g., a human opsin proximal promoter). In some embodiments, the promoter comprises a sequence that is at least 80%, at least 85%, at least 90%, at least 91%, at least 92%, at least 93%, at least 94%, at least 95%, at least 96%, at least 97%, at least 98%, at least 99%, or 100% identical to SEQ ID NO: 12 below.

In some embodiments, the promoter driving shRNA expression comprises a sequence that is at least 90%, at least 91%, at least 92%, at least 93%, at least 94%, at least 95%, at least 96%, at least 97%, at least 98%, at least 99%, or 100% identical to SEQ ID NO: 13 below. In some embodiments, the promoter driving shRNA expression comprises a sequence that is at least 90%, at least 91%, at least 92%, at least 93%, at least 94%, at least 95%, at least 96%, at least 97%, at least 98%, at least 99%, or 100% identical to SEQ ID NO: 14 below.

The sequences of the exemplary promoters are as follows:

VMD2 promoter, 623 bp fragment
(SEQ ID NO: 12)
AATTCTGTCATTTTACTAGGGTGATGAAATTCCCA
AGCAACACCATCCTTTTCAGATAAGGGCACTGAGG
CTGAGAGAGGAGCTGAAACCTACCCGGCGTCACCA
CACACAGGTGGCAAGGCTGGGACCAGAAACCAGGA
CTGTTGACTGCAGCCCGGTATTCATTCTTTCCATA
GCCCACAGGGCTGTCAAAGACCCCAGGGCCTAGTC
AGAGGCTCCTCCTTCCTGGAGAGTTCCTGGCACAG
AAGTTGAAGCTCAGCACAGCCCCCTAACCCCCAAC
TCTCTCTGCAAGGCCTCAGGGGTCAGAACACTGGT
GGAGCAGATCCTTTAGCCTCTGGATTTTAGGGCCA
TGGTAGAGGGGGTGTTGCCCTAAATTCCAGCCCTG
GTCTCAGCCCAACACCCTCCAAGAAGAAATTAGAG
GGGCCATGGCCAGGCTGTGCTAGCCGTTGCTTCTG
AGCAGATTACAAGAAGGGACCAAGACAAGGACTCC
TTTGTGGAGGTCCTGGCTTAGGGAGTCAAGTGACG
GCGGCTCAGCACTCACGTGGGCAGTGCCAGCCTCT
AAGAGTGGGCAGGGGCACTGGCCACAGAGTCCCAG
GGAGTCCCACCAGCCTAGTCGCCAGACC
H1 promoter
(SEQ ID NO: 13)
TAAAACGACGGCCAGTGAATTCATATTTGCATGTC
GCTATGTGTTCTGGGAAATCACCATAAACGTGAAA
TGTCTTTGGATTTGGGAATCTTATAAGTTCTGTAT
GAGACCACT
U6 promoter
(SEQ ID NO: 14)
GAGGGCCTATTTCCCATGATTCCTTCATATTTGCA
TATACGATACAAGGCTGTTAGAGAGATAATTGGAA
TTAATTTGACTGTAAACACAAAGATATTAGTACAA
AATACGTGACGTAGAAAGTAATAATTTCTTGGGTA
GTTTGCAGTTTTAAAATTATGTTTTAAAATGGACT
ATCATATGCTTACCGTAACTTGAAAGTATTTCGAT
TTCTTGGCTTTATATATCTTGTGGAAAGGAC

In some embodiments, the BEST1 coding sequence is in a vector, such as an AAV vector or plasmid.

In some embodiments, the vector as described herein comprises a sequence that is at least 80%, at least 85%, at least 90%, at least 91%, at least 92%, at least 93%, at least 94%, at least 95%, at least 96%, at least 97%, at least 98%, at least 99%, or 100% identical to a de-targeted BEST1 sequence, SEQ ID NO: 10.

In some embodiments, a vector encoding a functional BEST1 sequence is provided to supplement or correct (e.g., at least partially) cellular BEST1 function without knocking down endogenous BEST1 gene expression. In some embodiments, the BEST1 sequence is codon-optimized.

In some embodiments, a vector encoding a functional BEST1 sequence and an shRNA sequence is provided to supplement or correct (e.g., at least partially) cellular BEST1 function and knock down endogenous BEST1 gene expression. In some embodiments, endogenous Best1 expression is knocked down using shRNA. In some embodiments, the BEST1 sequence is codon-optimized. In some embodiments, the BEST1 sequence is modified to be resistant to the shRNA. In some embodiments, the BEST1 and shRNA sequences are encoded on the same AAV vector.

In some embodiments, the disclosure provides a method of modulating BEST1 expression in a subject, the method comprising administering to the subject, such as a human subject, a composition comprising a vector or rAAV particle and a pharmaceutically acceptable carrier. In some aspects, the disclosure provides a method of treating bestrophinopathies (e.g., Best Disease and ARB) in a subject, the method comprising administering a composition.

In some embodiments, the disclosure provides a composition for use in treating Best Disease and a composition for use in the manufacture of a medicament to treat Best Disease. In some aspects, the disclosure provides a composition comprising a vector or rAAV particle, wherein the vector encodes a functional BEST1 sequence, for use in treating ARB and a composition for use in the manufacture of a medicament to treat ARB.

Aspects of the disclosure relate to recombinant adeno-associated virus (rAAV) particles for delivery of an rAAV vector as described herein (e.g., encoding an shRNA and/or a replacement BEST1) into various tissues, organs, and/or cells. In some embodiments, the rAAV particles comprise a capsid protein as described herein, e.g., an AAV2 capsid protein. In some embodiments, the vector contained within the rAAV particle encodes an RNA of interest (e.g., an shRNA comprising the sequence of SEQ ID NO: 1) and comprises a replacement BEST1 coding sequence (e.g., comprising the sequence of SEQ ID NO: 10).

Recombinant AAV (rAAV) vectors contained within an rAAV particle may comprise at a minimum (a) one or more heterologous nucleic acid regions (e.g., encoding an shRNA and/or a Best1 protein) and (b) one or more regions comprising inverted terminal repeat (ITR) sequences (e.g., wild-type ITR sequences or engineered ITR sequences) flanking the one or more heterologous nucleic acid regions (or transgenes). In some embodiments, the heterologous nucleic acid region encodes an RNA of interest (e.g., an shRNA comprising the sequence of SEQ ID NO: 3) and comprises a replacement BEST1 coding sequence (e.g., comprising the sequence of SEQ ID NO: 10). In some embodiments, the rAAV vector is between 4 kb and 5 kb in size (e.g., 4.2 to 4.7 kb in size). This rAAV vector may be encapsidated by a viral capsid, such as an AAV2 capsid. In some embodiments, the rAAV vector is single-stranded. In some embodiments, the rAAV vector is double-stranded. In some embodiments, a double-stranded rAAV vector may be, for example, a self-complementary vector that contains a region of the vector that is complementary to another region of the vector, initiating the formation of the double-strandedness of the vector.

As disclosed herein, analysis of Best1 structure with targeted mutations has shown that loss of retinal pigment epithelium apical microvilli and resulting microdetachment of the retina represent the earliest features of canine bestrophinopathies. Retinal light exposure expands, and dark adaptation contracts, the microdetachments. Subretinal adeno-associated virus-based gene therapy corrects both the vitelliform lesions and the light-modulated microdetachments.

Studies of molecular pathology in the canine BEST1 disease model revealed retina-wide abnormalities at the RPE-PR interface associated with defects in the RPE microvillar ensheathment and a cone PR-associated insoluble interphotoreceptor matrix. In vivo imaging demonstrated a retina-wide RPE-PR microdetachment, which contracted with dark adaptation and expanded upon exposure to a moderate intensity of light.

Subretinal BEST1 gene augmentation therapy using adeno-associated virus 2 reversed not only clinically detectable subretinal lesions but also the diffuse microdetachments. Immunohistochemical analyses showed correction of the structural alterations at the RPE-PR interface in areas with BEST1 transgene expression. Successful treatment effects were demonstrated in three different canine BEST1 genotypes with vector titers in the 0.1×10¹¹ to 5×10¹¹ vector genomes per mL range. Patients with biallelic BEST1 mutations exhibited large regions of retinal lamination defects, severe PR sensitivity loss, and slowing of the retinoid cycle. Human translation of canine BEST1 gene therapy success in reversal of macro- and microdetachments through restoration of cytoarchitecture at the RPE-PR interface has promise to result in improved visual function and prevent disease progression in patients affected with bestrophinopathies.

As further disclosed herein, it was discovered that adeno-associated virus (AAV)2-mediated BEST1 gene augmentation corrects this primary subclinical defect as well as the disease.

The rAAV particle may be of any AAV serotype, including any derivative or pseudotype (e.g., 1, 2, 3, 4, 5, 6, 7, 8, 9, 10, 2/1, 2/5, 2/8, or 2/9). As used herein, the serotype of an rAAV particle refers to the serotype of the capsid proteins. In some embodiments, the rAAV particle is AAV2. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. Such AAV serotypes and derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan Al, Schaffer D V, Samulski R J.). In some embodiments, the rAAV particle is a pseudotyped rAAV particle, which comprises (a) a nucleic acid vector comprising ITRs from one serotype (e.g., AAV2) and (b) a capsid comprised of capsid proteins derived from another serotype (e.g., AAV5). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of producing rAAV particles and rAAV vectors are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Nos. US 2007/0015238 and US 2012/0322861, which are incorporated herein by reference; and plasmids and kits available from ATCC and Cell Biolabs, Inc.). For example, a plasmid containing the rAAV vector may be combined with one or more helper plasmids, e.g., that contain a rep gene (e.g., encoding Rep78, Rep68, Rep52 and Rep40) and a cap gene (e.g., encoding VP1, VP2, and VP3, including a modified VP3 region as described herein), and transfected into a producer cell line such that the rAAV particle can be packaged and subsequently purified.

In some embodiments, the one or more helper plasmids include a first helper plasmid comprising a rep gene and a cap gene (e.g., encoding a rAAV capsid protein as described herein) and a second helper plasmid comprising a E1a gene, a E1b gene, a E4 gene, a E2a gene, and a VA gene. In some embodiments, the rep gene is a rep gene derived from AAV2 and the cap gene is derived from AAV2 and may include modifications to the gene in order to produce the modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG (R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca; and Addgene, Cambridge, Mass.; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

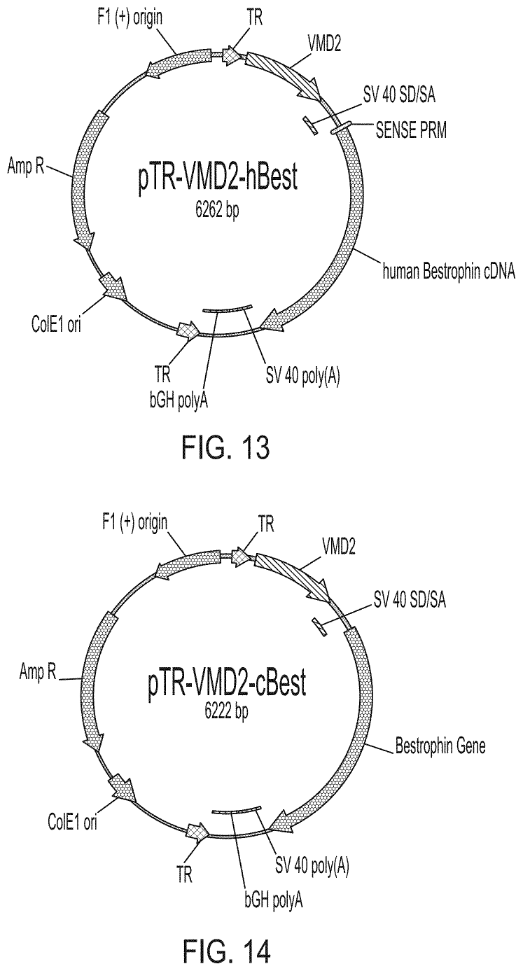

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a heterologous nucleic acid vector described herein (e.g. a plasmid containing a heterologous nucleic acid comprising wild-type or mutant cBEST1 or hBEST1 gene shown in FIG. 13, 14, 15 or 16). The HEK293 cells are then incubated for at least 60 hours to allow for rAAV particle production. Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

The disclosure also contemplates host cells that comprise an shRNA, a vector, or an rAAV particle as described herein. Such host cells include mammalian host cells, with human host cells being preferred, and may be isolated, e.g., in cell or tissue culture. In some embodiments, the host cell is a cell of the eye.

In some aspects, the disclosure provides formulations of one or more rAAV-based compositions disclosed herein in pharmaceutically acceptable solutions for administration to a cell or an animal, either alone or in combination with one or more other modalities of therapy, and in particular, for therapy of human cells, tissues, and diseases affecting man.

Accordingly, in some embodiments, a composition is provided which comprises an shRNA, a vector, or an rAAV particle as described herein and optionally a pharmaceutically acceptable carrier. In some embodiments, the compositions described herein can be administered to a subject in need of treatment. In some embodiments, the subject has or is suspected of having one or more conditions, diseases, or disorders of the brain and/or eye (e.g., Best Disease). In some embodiments, the subject has or is suspected of having one or more of the conditions, diseases, and disorders disclosed herein (e.g., Best Disease). In some embodiments, the subject has one or more endogenous mutant BEST1 alleles (e.g., associated with or that cause a disease or disorder of the eye or retina). In some embodiments, the subject has at least one autosomal dominant mutant BEST1 allele (e.g., that causes Best Disease). In some embodiments, the subject is a human. In some embodiments, the subject is a non-human primate. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. Other exemplary subjects include domesticated animals such as dogs and cats; livestock such as horses, cattle, pigs, sheep, goats, and chickens; and other animals such as mice, rats, guinea pigs, and hamsters.

In some embodiments, the dose of rAAV particles administered to a cell or a subject may be on the order ranging from 10⁶ to 10¹⁴ particles/mL or 10³ to 10¹⁵ particles/mL, or any values therebetween for either range, such as for example, about 10⁶, 10⁷, 10⁸, 10⁹, 10¹⁰, 10¹¹, 10¹², 10¹³, or 10¹⁴ particles/mL. In one embodiment, rAAV particles of higher than 10¹³ particles/mL are be administered. In some embodiments, the dose of rAAV particles administered to a subject may be on the order ranging from 10⁶ to 10¹⁴ vector genomes (vgs)/mL or 10³ to 10¹⁵ vgs/mL, or any values therebetween for either range, such as for example, about 10⁶, 10⁷, 10⁸, 10⁹, 10¹⁰, 10¹¹, 10¹², 10¹³, or 10¹⁴ vgs/mL. In one embodiment, rAAV particles of higher than 10¹³ vgs/mL are be administered. The rAAV particles can be administered as a single dose, or divided into two or more administrations as may be required to achieve therapy of the particular disease or disorder being treated. In some embodiments, 0.0001 mL to 10 mLs (e.g., 0.0001 mL, 0.001 mL, 0.01 mL, 0.1 mL, 1 mL, 10 mLs) are delivered to a subject in a dose.

In some embodiments, rAAV particle titers range from 1×10¹⁰ to 5×10¹³ vg/ml. In some embodiments, rAAV particle titers can be about 1×10¹⁰, 2.5×10¹⁰, 5×10¹⁰, 1×10¹¹, 2×10¹¹, 2.5×10¹¹, 5×10¹¹, 1×10¹², 2.5×10¹², 5×10¹², 1×10¹³, 2.5×10¹³, or 5×10¹³ vg/mL. In some embodiments, particle titers are less than 1×10¹⁰ vg/mL. In some embodiments, rAAV particle titers are greater than 1×10¹⁵ vg/mL. In some embodiments, rAAV particle titers are greater than 5×10¹³ vgs/mL. In particular embodiments, rAAV particle titers are about 2×10¹¹ or 2.5×10¹¹. In some embodiments, rAAV particles are administered via methods further described herein (e.g., subretinally or intravitreally).

The rAAV particles can be administered as a single dose, or divided into two or more administrations as may be required to achieve therapy of the particular disease or disorder being treated. In some embodiments, from 1 to 500 microliters of a composition (e.g., comprising an rAAV particle) described in this application is administered to one or both eyes of a subject. For example, in some embodiments, about 1, about 10, about 50, about 100, about 200, about 300, about 400, or about 500 microliters can be administered to each eye. However, it should be appreciated that smaller or larger volumes could be administered in some embodiments.

If desired, rAAV particle or nucleic acid vectors may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles may thus be delivered along with various other agents as required in the particular instance. Such compositions may be purified from host cells or other biological sources, or alternatively may be chemically synthesized as described herein.

In other aspects, the disclosure provides formulations of one or more of the plasmids encoding an shRNA as disclosed herein in pharmaceutically acceptable solutions for administration to a cell or an animal, either alone or in combination with one or more other modalities of therapy, and in particular, for therapy of human cells, tissues, and diseases affecting man. The disclosure also provides methods of administration of plasmids encoding an shRNA as disclosed herein. Exemplary methods comprised methods of administration of plasmids to mammals, e.g. humans.

In some embodiments, the disclosed plasmid formulations for administration to mammals (e.g., humans) comprise DNA plasmid vector in phosphate buffered saline (PBS). The concentration of the vector may be between 1 mg/ml and 3 mg/ml. In certain embodiments, the concentration is about 2 mg/ml. In other embodiments, the concentration is about 1.6 mg/ml, about 1.7 mg/ml, about 1.75 mg/ml, about 1.8 mg/ml, about 1.85 mg/ml, about 1.9 mg/ml, about 1.95 mg/ml, about 2.05 mg/ml, about 2.1 mg/ml, or about 2.15 mg/ml.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intra-articular, and intramuscular administration and formulation.

Typically, these formulations may contain at least about 0.1% of the therapeutic agent (e.g., rAAV particle or plasmid) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., rAAV particle) in each therapeutically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver an shRNA, a vector, or an rAAV particle as described herein in suitably formulated pharmaceutical compositions disclosed herein, either subcutaneously, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection.

The pharmaceutical forms of compositions (e.g., comprising an shRNA, a vector, or an rAAV particle as described herein) suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The term “carrier” refers to a diluent, adjuvant, excipient, or vehicle with which the shRNA, vector, or rAAV particle as described herein is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers.

The compositions of the present disclosure can be delivered to the eye through a variety of routes. They may be delivered intraocularly, by topical application to the eye or by intraocular injection into, for example the vitreous (intravitreal injection) or subretinal (subretinal injection) inter-photoreceptor space. In some embodiments, they are delivered to rod photoreceptor cells. Alternatively, they may be delivered locally by insertion or injection into the tissue surrounding the eye. They may be delivered systemically through an oral route or by subcutaneous, intravenous or intramuscular injection. Alternatively, they may be delivered by means of a catheter or by means of an implant, wherein such an implant is made of a porous, non-porous or gelatinous material, including membranes such as silastic membranes or fibers, biodegradable polymers, or proteinaceous material. They can be administered prior to the onset of the condition, to prevent its occurrence, for example, during surgery on the eye, or immediately after the onset of the pathological condition or during the occurrence of an acute or protracted condition.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions may be prepared by incorporating an shRNA, a vector, or an rAAV particle as described herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The amount of composition (e.g., comprising an shRNA, a vector, or an rAAV particle as described herein) and time of administration of such composition will be within the purview of the skilled artisan having benefit of the present teachings. It is likely, however, that the administration of therapeutically-effective amounts of the disclosed compositions may be achieved by a single administration, such as for example, a single injection of sufficient numbers of rAAV particles to provide therapeutic benefit to the patient undergoing such treatment. Alternatively, in some circumstances, it may be desirable to provide multiple, or successive administrations of the composition, either over a relatively short, or a relatively prolonged period of time, as may be determined by the medical practitioner overseeing the administration of such compositions.

In some embodiments, rod cells remain structurally intact and/or viable upon silencing of cellular BEST1 gene expression. In some embodiments, rod cells in which cellular BEST1 gene expression is silenced may have shortened outer segments which would normally contain BEST1. In some embodiments, the length of the outer segments can be maintained or restored (e.g., partially or completely) using the exogenously added (hardened) BEST1 gene, the expression of which is resistant to silencing using the compositions described in this application.

To “treat” a disease as the term is used herein, means to reduce the frequency or severity of at least one sign or symptom of a disease or disorder experienced by a subject (e.g., Best Disease). The compositions described above are typically administered to a subject in an effective amount, that is, an amount capable of producing a desirable result. The desirable result will depend upon the active agent being administered. For example, an effective amount of a rAAV particle may be an amount of the particle that is capable of transferring a heterologous nucleic acid to a host organ, tissue, or cell.

Toxicity and efficacy of the compositions utilized in methods of the disclosure can be determined by standard pharmaceutical procedures, using either cells in culture or experimental animals to determine the LD50 (the dose lethal to 50% of the population). The dose ratio between toxicity and efficacy the therapeutic index and it can be expressed as the ratio LD50/ED50. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED50 with little or no toxicity. The dosage may vary within this range depending upon the dosage form employed and the route of administration utilized.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

### Early Retina-Wide Pathology at the RPE-PR Interface.

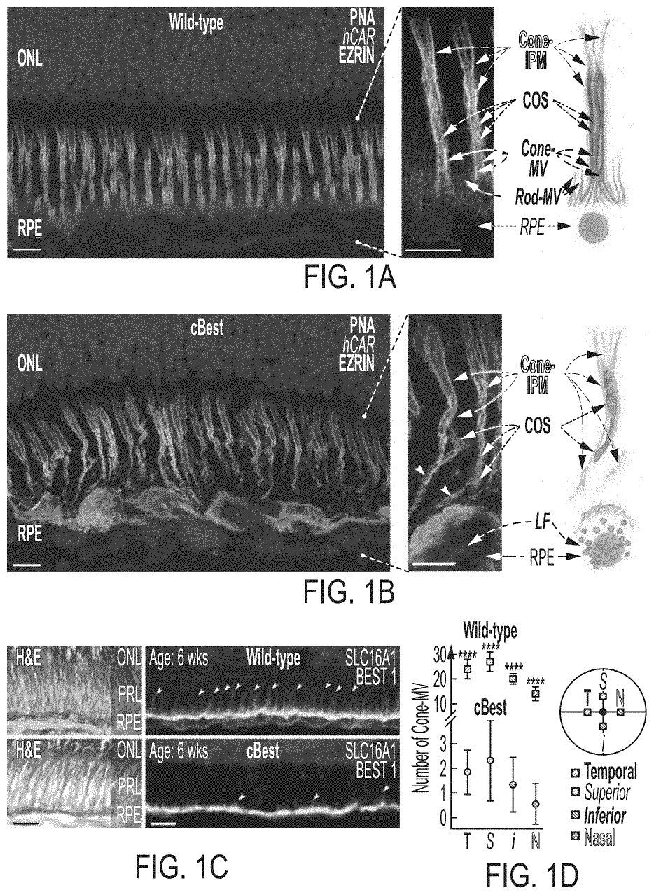

To understand the pathophysiology behind the impaired RPE-PR interaction, cBest retinas with clinically obvious disease were evaluated. The key features of RPE apical membrane responsible for direct interaction with PR OS s were examined by immunohistochemistry (IHC) against EZRIN, a membrane-cytoskeleton linker protein essential for formation of RPE apical MV, and combined with human cone arrestin (hCAR) and peanut agglutinin lectin (PNA) labeling to distinguish the cone PR matrix-specific interface. Confocal microscopy and analysis of 3D reconstruction images from the wild-type (WT) retina exposed a complex sheet-like structure of both inherent constituents of RPE apical membrane: cone- and rod-associated MV. Cone-MV (also known as RPE apical cone sheath) were more pronounced than rod-MV, and formed a highly organized wrapping that tethered individual cone outer segments (COSs) to the RPE apical surface (FIG. 1A). In the subretinal space, this intercellular complex was further encased with an equally intricate cone-specific insoluble extracellular matrix sheath (cone-IPM) detected by selective binding of PNA lectin (FIG. 1A). In diseased cBest retinas, however, such complex extracellular compartmentalization of COSs was lost, and the dearth of microvillar ensheathment was accompanied by hypertrophied RPE cells overloaded with lipofuscin granules and compromised insoluble cone-IPM (FIG. 1B). These observations were confirmed in three distinct cBEST1 genotypes (R25*/R25*, P463fs/P463fs, and R25*/P463fs) examined across both the tapetal and nontapetal portions of the retina in 22 eyes after disease onset (age range 45 to 270 wk).

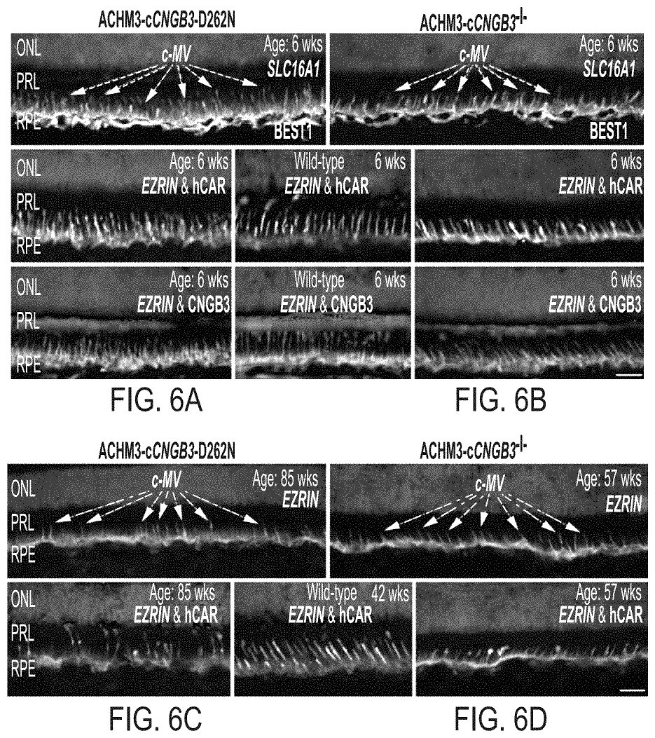

To evaluate the possibility that the structural cone-MV abnormalities are secondary to cone dysfunction and disease, the RPE-COS interaction in a different canine IRD model was examined: a primary cone photoreceptor channelopathy, CNGB3-associated achromatopsia. The RPE-COS complex was first examined at 6 wk of age; CNGB3-mutant retinas, harboring either a missense or locus deletion mutation, showed no apparent irregularities at the RPE-PR interface, and the proper localization of RPE apical markers was associated with specific anti-BEST1 labeling (FIGS. 6A and 6B). Double immunostaining demonstrated specific distribution of EZRIN along cone-MV interdigitating with hCAR-positive yet CNGB3-negative COSs. As a consequence of CNGB3 channel subunit dysfunction in older (ages 57 and 85 wk) mutant retinas, which undergo a gradual cone PR degeneration, it was found that the microvillar ensheathment of the RPE apical domain still remained largely intact (FIGS. 6C and 6D).

The findings in CNGB3-mutant retinas suggested that the structural alterations associated with cone-MV ensheathment in cBest were not secondary to a cone defect but specific to the RPE channelopathy triggered by mutations in BEST1. The 6-week time point, which is well before clinical disease onset and near the end of postnatal retinal differentiation in dogs, was the focus of these experiments (FIGS. 1C and 1D). In contrast to the age-matched WT control, the lack of specific basolateral BEST1 immunolabeling in the cBest RPE was associated with a rather smooth apical surface and clearly underdeveloped (vestigial) apical microvilli (FIG. 1C, arrowheads). Quantification of the spatial density of cone-MV and the length of cone- and rod-MV was performed on deconvolved 3D Z-stack projection images at four retinal locations (FIG. 1D). Significant differences (P<0.0001) in the mean number of cone-MV were found between cBest and WT in each retinal region examined (FIG. 1D). Even though the cone photoreceptor numbers were comparable to controls, the cone-MV in cBest were much fewer in number, sparsely distributed, and consistently appeared shorter and much finer than those in controls regardless of the topographical location. In control (WT) eyes, the average length of cone-MV was 17.4 (±0.25) μm in the tapetal superotemporal quadrant and 12.3 (±0.23) μm in the inferior nontapetal retina, whereas the length of rod-MV was 6.7 (±0.11) μm and 5.3 (±0.27) μm in the tapetal and nontapetal portions of the retina, respectively. In cBest, however, the average length of rare cone-MV extensions identified was substantially reduced (6.0±0.31 and 6.5±0.74 μm in the central tapetal and nontapetal inferior parts, respectively). A quantitative assessment of the minute rod-MV in cBest was beyond the limits of optical resolution.

cBEST1-Mutant Eyes have Retina-Wide Microdetachments that Expand with Light Exposure.

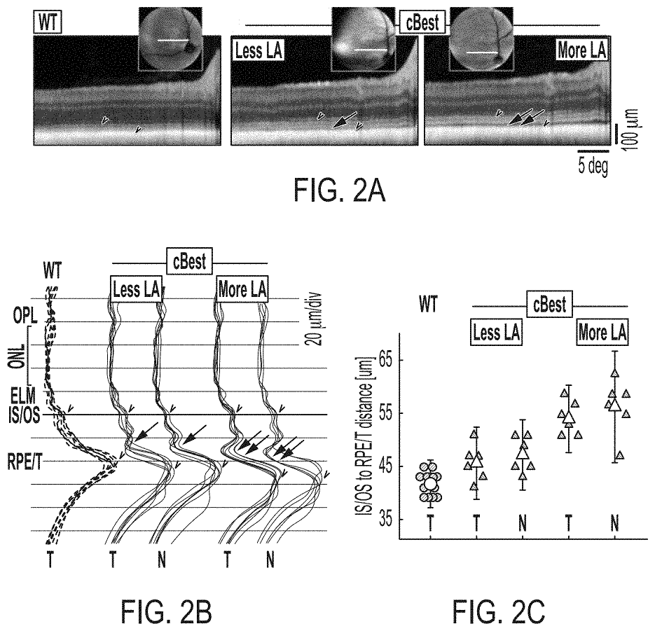

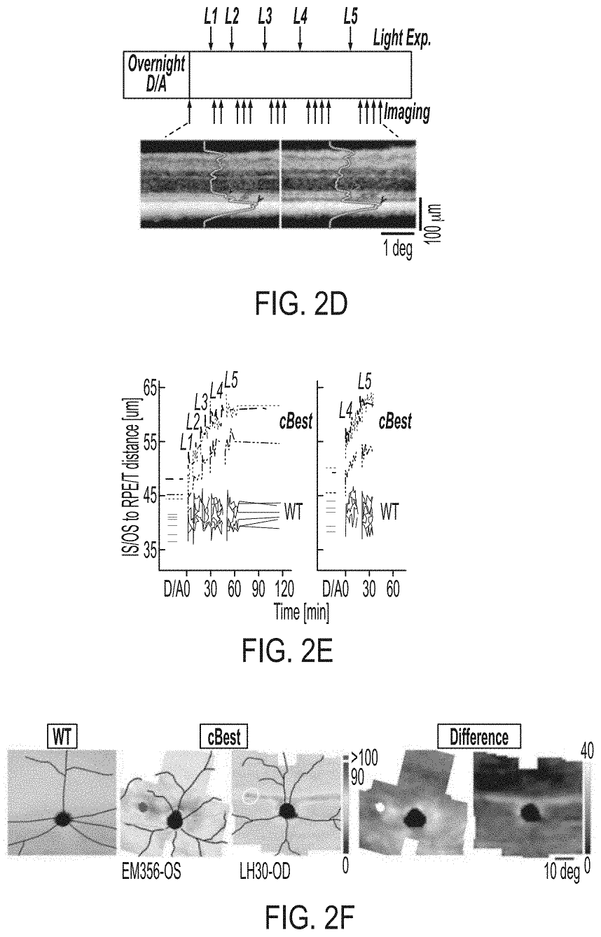

To determine in vivo correlates of the early RPE-PR interface abnormalities detected by IHC, noninvasive imaging with optical coherence tomography (OCT) was used to evaluate cBest eyes at young ages, well before ophthalmoscopic lesions are detectable. Qualitatively, central retinas of all evaluated eyes showed an additional hyposcattering layer in the outer retina located distal to the outer nuclear layer (ONL) that was not detectable in WT eyes (FIG. 2A, arrow and double arrow). Unexpectedly, the hyposcattering layer was variable with repeated recordings in the same eye within a single experimental session. Further analyses uncovered that the width of the hyposcattering layer was greater in scans obtained toward the end of an imaging session, when the retina would have been exposed to greater retinal irradiance due to intervening autofluorescence imaging performed with bright short-wavelength lights (FIG. 2A, double arrow, more LA). The width of the hyposcattering layer was less in scans obtained early in the imaging session before autofluorescence imaging was performed (FIG. 2A, arrow, less LA).

Quantitative studies were performed by obtaining longitudinal reflectivity profiles and making measurements both at nasal and temporal retinal locations. WT eyes (n=12, ages 15 to 17 wk) showed outer retinal hyperscattering peaks at the outer plexiform layer (OPL) and the external limiting membrane (ELM) defining the intervening hyposcattering layer as the ONL (FIG. 2B). Distal to the ELM was a hyperscattering peak corresponding to the junction between inner and outer segments of photoreceptors (IS/OS), a major peak originating near the RPE-tapetum interface (RPE/T), and an intervening minor hyperscattering peak corresponding to photoreceptor OS tips, which was often difficult to resolve (FIG. 2B). An abnormal hyposcattering layer (FIG. 2B, arrows) was detectable in cBest eyes (n=6, age 11 wk) with less light exposure. With greater light exposure, the hyposcattering layer became deeper and more distinct (FIG. 2B, double arrows); both nasal and temporal retinal locations showed the same effect. The distance between the IS/OS and RPE/T peaks (FIGS. 2A and 2B, arrowheads) was measured. In WT eyes, the distance was 41.3 (±4.5) μm, whereas in cBest eyes this distance was significantly greater (P<0.001) at 46.8 (±6.7) μm and 45.2 (±6.8) μm (less light exposure) and 55.8 (±10.5) μm and 53.5 (±6.3) μm (more light exposure) for nasal and temporal retinal regions, respectively (FIG. 2C).

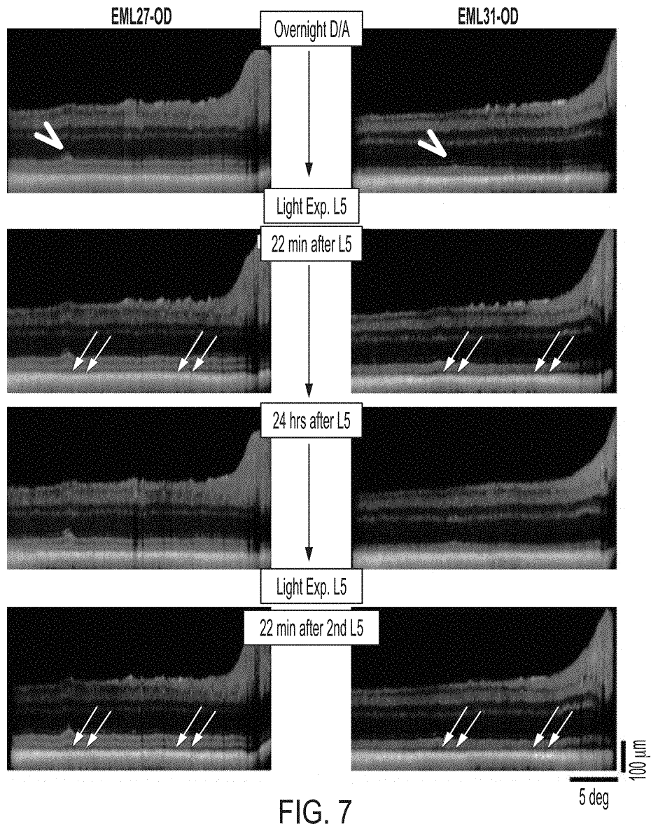

Two types of experiments were performed to better understand the thickness of the hyposcattering layer as a function of light exposure. In the main experiment (WT, n=12, age 15 to 17 wk; cBest, n=3, age 13 wk), eyes were dark-adapted overnight and then sequential imaging was performed in the dark over a 2-h period with five intervening brief 488-nm light exposures of incrementally greater intensity ranging from very dim lights to moderate lights produced by standard clinical ophthalmic equipment (FIG. 2D). In a shorter experimental protocol, only the highest two light exposures were used in different eyes (cBest, n=3, age 13 wk). After overnight dark adaptation, IS/OS-RPE/T distance was 40.0 (±4.5) μm in WT eyes, whereas it was 47.1 (±4.8) μm in cBest (FIG. 2E); the difference was statistically significant (P<0.001). Increasingly brighter light exposures resulted in monotonic expansion of the IS/OS-RPE/T distance in cBest eyes, reaching an apparent plateau of 59.4 (±8.7) μm (FIG. 2E). In WT eyes, the effect of the light exposure was either negligible or small, with the IS/OS-RPE/T distance reaching a plateau of 40.9 (±4.3) μm. Thus, exposure to light appeared to cause an acute retinal microdetachment of up to 18.4 (±8.7) μm in cBest eyes within minutes at an age preceding any detectable ophthalmoscopic findings. The light-mediated microdetachment disappeared over a time span of less than 24 h (FIG. 7).

In preparation for localized gene therapy, retinotopic distribution of light-driven microdetachments was evaluated in fully light-adapted cBest and WT eyes (FIG. 2F). The mean IS/OS-RPE/T distance across WT eyes (n=4, age 104 wk) was relatively homogeneous across superior and inferior retinal areas, with a clear boundary corresponding to the transition between tapetal and pigmented (nontapetal) retina. Greater distance in the tapetal retina of WT eyes was likely due to differences in the dominant contributors to the hyperscattering peak (tapetum in the tapetal retina versus pigmented RPE). In a cBest eye (R25*/P463fs) at age 297 wk, there was a relatively diffuse retina-wide microdetachment in addition to grossly obvious retinal detachment at the fovea-like region (FIG. 2F, demarcated with darker color). In a younger cBest eye (P463fs/P463fs) at age 12 wk, there was a distinct band of greater microdetachment along the visual streak and surrounding the optic nerve head even though no ophthalmoscopic abnormalities were evident. Difference maps between mutant eyes and mean WT demonstrated the spatial distribution of the extent of microdetachments (FIG. 2F, Right).

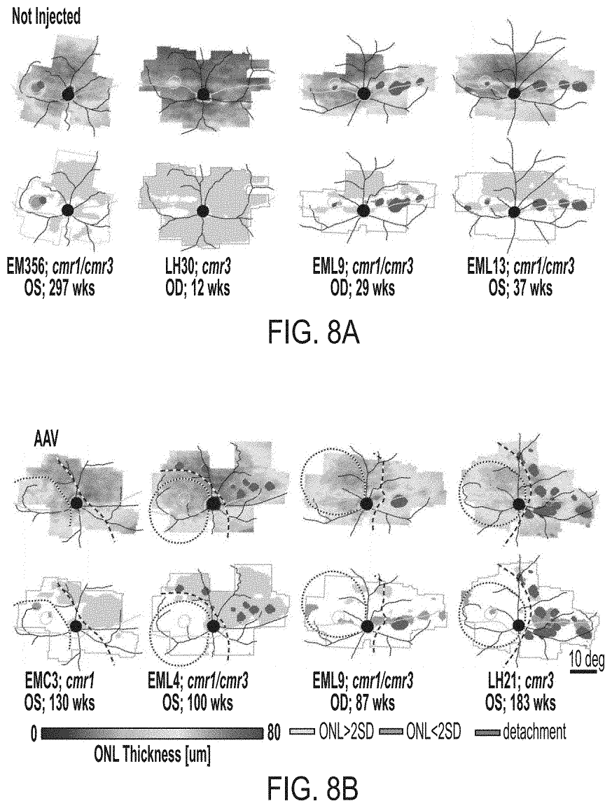

To assess the potential adverse consequences on photoreceptors, ONL thickness was topographically mapped across the retinal areas with microdetachments (FIGS. 8A and 8B). The microdetachments did not result in thinning of the ONL that would be expected from photoreceptor degeneration. Instead, there was a tendency for the ONL in cBest to be homogeneously thicker than WT; hyperthick regions typically included the central-superior tapetal retina, but could also extend into the inferior nontapetal retina (FIG. 8A). Of importance, the hyperthick regions of the ONL, when examined microscopically, had numbers of PR nuclei that were comparable to controls. This suggests an expansion of internuclear spacing as the likely cause of hyperthick ONL observed by imaging.

### Natural History of Canine Bestrophinopathy.

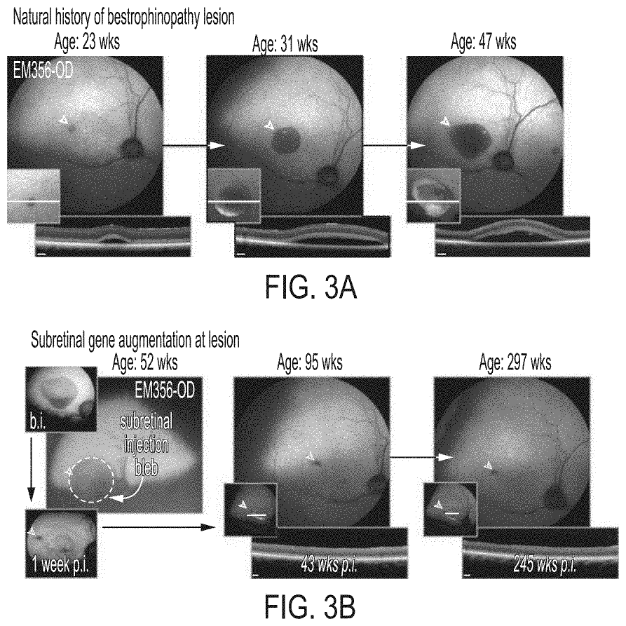

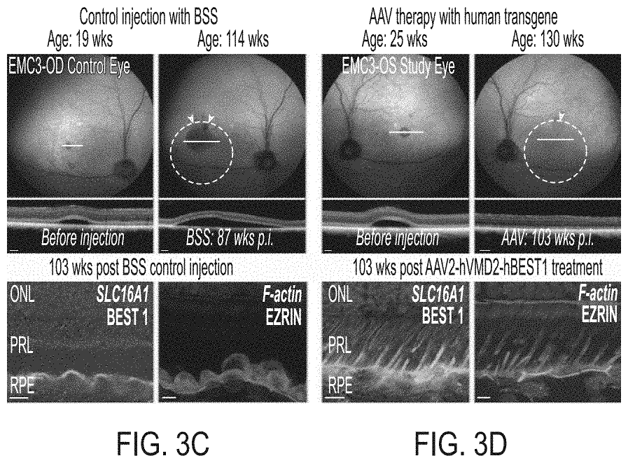

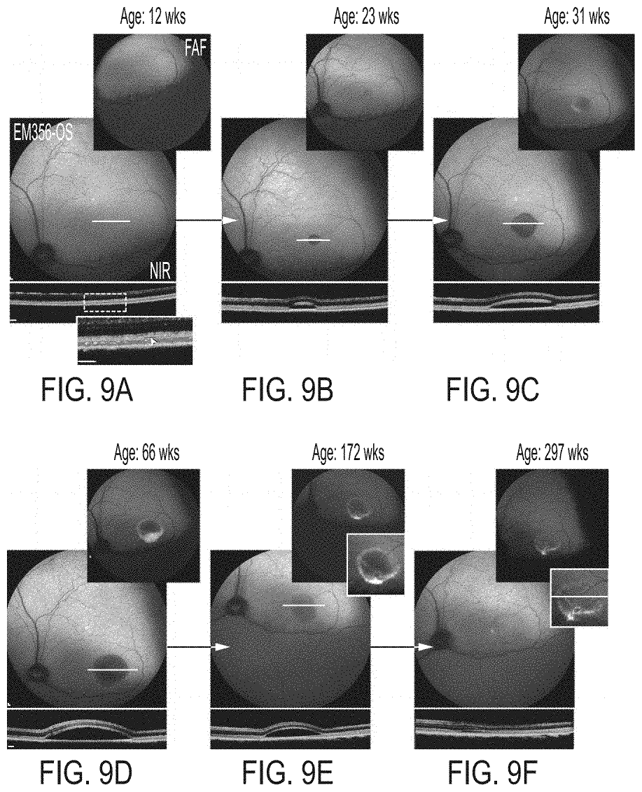

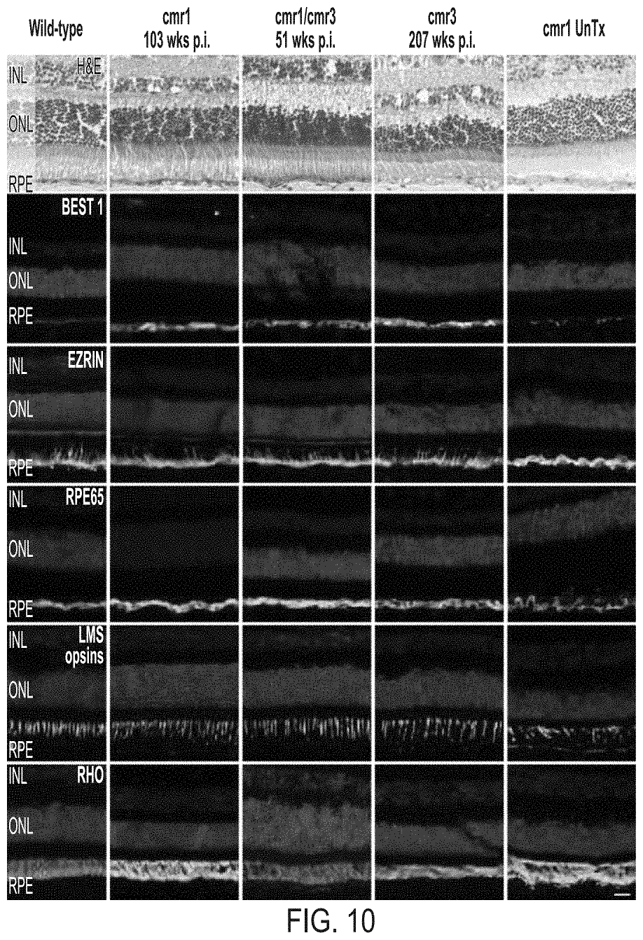

As a prerequisite to assessing gene therapy outcomes, the natural history of cBest was determined from a group of 18 dogs [12 male (M) and 6 female (F); age range 6 to 297 wk] (Table 1). cBest dogs were serially monitored by ophthalmoscopy and noninvasive imaging to detect the onset of earliest disease and understand disease progression. Based on the systematic in vivo imaging, the first disease signs were detected as early as 11 wk of age (mean age of 15 wk) as a subtle focal retinal elevation of the canine fovea-like regions (FIG. 9A). This discrete separation of photoreceptor layer from the underlying RPE progressed to form a larger subretinal macrodetachment (vitelliform lesion) evident en face and on the corresponding OCT scan at 23 wk of age (FIG. 9B). This discrete RPE-PR detachment on cross-sectional imaging, yet unnoticeable on en face imaging, was found to be consistent among cBest eyes examined (n=34), regardless of genotype. From the subclinical stage, the disease progressed to form a macrodetachment (vitelliform stage) limited to the canine fovea and surrounded by microdetachment (FIG. 3A, Left panel). The primary lesion gradually evolved to manifest as a characteristic bullous detachment within the area centralis that encompassed the fovea-like region (FIG. 3A, Middle and Right panels and FIGS. 9B-9D). The presence of distinctive hyperautofluorescence was evident in the inferior part of the lesion (FIG. 3A, Middle Inset panels; pseudohypopyon stage). The advanced disease stages involved a partial resorption and dispersion of the hyperautofluorescent material within the central lesion, associated with significant thinning of ONL (FIGS. 9E and 9F).

In each case followed by serial imaging (Table 1), cBest manifested bilaterally and nearly always presented a remarkable symmetry, albeit with a variable rate of progression (FIG. 3A and FIGS. 9A-9F). The gross retinal detachments, ophthalmoscopically visible in both eyes, either remained limited to the central retina or became more extensive with extracentral lesions scattered throughout, still with a strong predilection to the central cone-rich areas and associated with hyperthick ONL.

TABLE 1
Summary of AAV-BEST1-treated and control eyes used in the study.

| | | | | | | AAV titer | | Last | |
|---|---|---|---|---|---|---|---|---|---|
| | | cBEST1 | | | Age, | inj., | Volume | examination, | |
| Dog ID | Sex | status | Eye | Treatment | wk | vg/mL | inj., μL | wk p.i. | Outcome |
| EM356 | M | R25*/ | OD | AAV- | 52 | 1.5E+10 | 50 | 245 | Reversal |
| | | P463fs | | cBEST1 | | | | | |
| | | | OS | UnTx | | | | 245 | Progression |
| EM385 | M | R25*/ | OD | BSS | 39 | | 50 | 105 | Progression |
| | | R25* | | | | | | | |
| | | | OS | AAV- | 39 | 1.5E+11 | 60 | 105 | Reversal |
| | | | | cBEST1 | | | | | |
| EMC1 | F | R25*/ | OD | AAV- | 27 | 2.0E+11 | 110 | 156 | Reversal |
| | | R25* | | hBEST1 | | | | | |
| | | | OS | BSS | 27 | | 110 | 156 | Progression |
| EMC3 | F | R25*/ | OD | BSS | 27 | | 110 | 103 | Progression |
| | | R25* | | | | | | | |
| | | | OS | AAV- | 27 | 2.0E+11 | 120 | 103 | Reversal |
| | | | | hBEST1 | | | | | |
| EML4 | M | R25*/ | OD | BSS | 58 | | 150 | 79 | Progression |
| | | P463fs | | | | | | | |
| | | | OS | AAV- | 58 | 2.5E+11 | 95 | 79 | Reversal |
| | | | | hBEST1 | | | | | |
| EML6 | F | R25*/ | OD | AAV- | 43 | 2.0E+11 | 100 | 51 | Reversal |
| | | P463fs | | hBEST1 | | | | | |
| | | | OS | BSS | 43 | | 110 | 51 | Progression |
| EML9 | M | R25*/ | OD | AAV- | 69 | 5.0E+11 | 65 | 54 | Reversal |
| | | P463fs | | hBEST1 | | | | | |
| | | | OS | BSS | 69 | | 70 | 54 | Progression |
| EML13 | F | R25*/ | OD | UnTx | | | | 52 | Progression |
| | | P463fs | | | | | | | |
| | | | OS | AAV- | 45 | 1.5E+11 | 100 | 52 | Reversal |
| | | | | hBEST1 | | | | | |
| EML22 | | R25*/ | OD | AAV- | 27 | 3.5E+11 | 100 | 39 | Reversal |
| | | P463fs | | hBEST1 | | | | | |
| | | | OS | AAV- | 27 | 3.5E+11 | 100 | 39 | Reversal |
| | | | | hBEST1 | | | | | |
| LH15 | | P463fs/ | OD | AAV- | 65 | 2.5E+11 | 180 | 207 | Reversal |
| | | P463fs | | hBEST1 | | | | | |
| | | | OS | AAV- | 65 | 2.5E+11 | 50 | 207 | Reversal |
| | | | | hBEST1 | | | | | |
| LH21 | | P463fs/ | OD | BSS | 27 | | 50 | 207 | Progression |
| | | P463fs | | | | | | | |
| | | | OS | AAV- | 27 | 2.5E+11 | 50 | 207 | Reversal |
| | | | | hBEST1 | | | | | |
| LH30 | | P463fs/ | OD | AAV- | 31 | 2.5E+11 | 110 | 13 | Reversal |
| | | P463fs | | hBEST1 | | | | | |
| | | | OS | AAV- | 31 | 2.5E+11 | 110 | 13 | Reversal |
| | | | | hBEST1 | | | | | |
| AS277 | M | WT | OU | UnTx | 6 | | | | |
| | | | | control | | | | | |
| EML24 | M | R25*/ | OU | UnTx | 6 | | | | |
| | | P463fs | | control | | | | | |
| EML21 | M | R25*/ | OU | UnTx | 13 | | | | |
| | | P463fs | | control | | | | | |
| EML23 | F | R25*/ | OU | UnTx | 13 | | | | |
| | | P463fs | | control | | | | | |
| N306 | M | WT | OU | UnTx | 15 | | | | |
| | | | | control | | | | | |
| N307 | F | WT | OU | UnTx | 15 | | | | |
| | | | | control | | | | | |
| N308 | F | WT | OU | UnTx | 15 | | | | |
| | | | | control | | | | | |
| N300 | M | WT | OU | UnTx | 17 | | | | |
| | | | | control | | | | | |
| N301 | M | WT | OU | UnTx | 17 | | | | |
| | | | | control | | | | | |
| N302 | F | WT | OU | UnTx | 17 | | | | |
| | | | | control | | | | | |
| CCAN | M | WT | OU | UnTx | 37 | | | | |
| | | | | control | | | | | |
| CCGS | M | WT | OU | UnTx | 37 | | | | |
| | | | | control | | | | | |
| LH14 | M | WT | OU | UnTx | 42 | | | | |
| | | | | control | | | | | |
| EML31 | M | R25*/ | OD | UnTx | 43 | | | | |
| | | P463fs | | control | | | | | |
| EML27 | M | R25*/ | OD | UnTx | 52 | | | | |
| | | P463fs | | control | | | | | |
| EM346 | M | R25*/ | OS | UnTx | 89 | | | | |
| | | R25* | | control | | | | | |
| N284 | F | WT | OS | UnTx | 145 | | | | |
| | | | | control | | | | | |
| N269 | F | WT | OS | UnTx | 199 | | | | |
| | | | | control | | | | | |

Key: BSS, balanced salt solution; cBEST1, canine transgene; hBEST1, human transgene; Inj., injected; OD, right eye; OS, left eye; OU, bilateral; p.i., postinjection; UnTx, untreated; vg, vector genomes; WT, wild type.
cBEST1 mutations: R25*/R25*, p.Arg25Ter-homozygote; P463fs/P463fs, p.Pro463fs-homozygote; R25*/P463fs, p.Arg25Ter/p.Pro463fs-compound heterozygous.

### Subretinal BEST1 Gene Augmentation Therapy Stably Corrects Disease.

To evaluate proof of concept for AAV2-mediated subretinal gene augmentation therapy, 22 cBest eyes were injected [vector titers in the 0.1 to 5×10¹¹ vector genomes (vg) per mL range or balanced salt solution (BSS) control] at 27 to 69 wk (Table 1) with a canine (cBEST1) or human (hBEST1) transgene driven by the human VMD2 promoter. Maps of exemplary AAV vectors comprising hBEST1 and cBEST1 heterologous nucleic acids used to make the disclosed rAAV particles are shown in FIGS. 13 and 14, respectively. cBest dogs exhibiting different stages of focal or multifocal retinal detachments were either injected unilaterally with AAV leaving the fellow eye uninjected, or with AAV in one eye and control (BSS) injection in the contralateral eye; three cases manifesting multifocal disease were injected bilaterally with AAV targeting the superotemporal quadrant, while the retinal areas outside of the surgical bleb served as an internal control (Table 1).

A representative result is shown from a compound heterozygous (R25*/P463fs) dog displaying advanced central retinal detachment in the right eye (EM356-OD) (FIG. 3A) that underwent subretinal injection with cBEST1 at the age of 52 wk (FIG. 3B, Left panel), while the fellow eye was not injected (EM356-OS) (FIGS. 9A-9F). Both eyes were monitored clinically and by in vivo imaging. Disease reversal was first apparent in the injected eye at 4 wk postinjection (p.i.) and retained a sustained effect long-term, as illustrated at 43 and 245 wk p.i. (FIG. 3B). In this and other cases with advanced disease that presented extensive accumulation of autofluorescent material within the subretinal macrodetachments (n=13 eyes), hyperautofluorescent signals were still detectable for several months post AAV injection but gradually faded over time (FIG. 3B, Inset panels). Based on noninvasive imaging, both the focal as well as extracentral lesions within the AAV-BEST1-treated regions resolved 4 to 12 wk p.i., and localized retinal reattachments remained stable thereafter (Table 1). There was no evidence of inflammatory responses in any of the AAV-treated eyes, and the longitudinal in vivo evaluations did not reveal adverse effects on the RPE or neural retina.

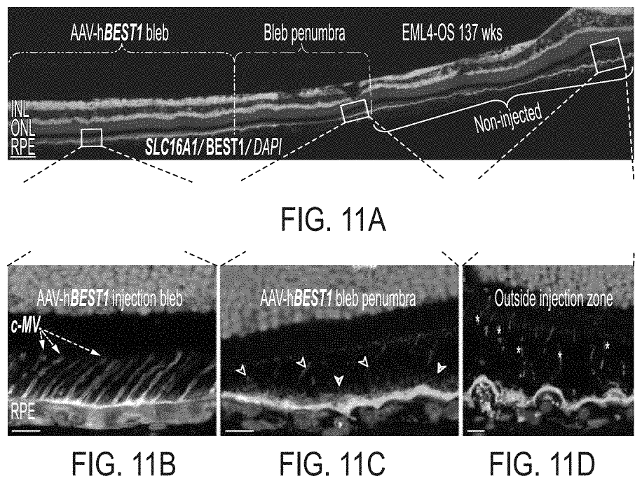

AAV-mediated treatment with the hBEST1 also resulted in lesion reversal and long-term disease correction (n=13 eyes). Representative in vivo imaging results and MC evaluations (FIGS. 3C and 3D) from a cBest dog (R25*/R25*) showed that the early bilateral lesions present before treatment disappeared after the study eye (EMC3-OS) was treated with AAV-hBEST1 (2×10¹¹ vg/mL) (FIG. 3D), while the lesion in the contralateral control eye (EMC3-OD) injected with BSS continued to enlarge (FIG. 3C). Based on funduscopic examination, in the illustrated example as well as in all other cases the transient retinal detachment associated with vector or BSS delivery resolved within 24 to 48 h p.i.; however, the retinal lesions injected with BSS reappeared as early as 1 wk p.i., and progressed along the natural disease course (FIG. 3C). This was in sharp contrast to the AAV-treated eyes, where both the early as well as more advanced lesions resolved within the first 6 wk after hBEST1 gene therapy, and the treated areas thereafter remained disease-free (FIG. 3D). Ophthalmological examinations and IHC assessments using RPE- and PR-specific markers showed no adverse effects on the retina up to 207 wk p.i. (FIGS. 3, 10 and 11). Of particular importance, the assessments of the retinal preservation p.i. revealed a remarkable restoration of retinal architecture at the RPE-PR interface, including extension of cone-MV and actin cytoskeleton rescue, corresponding to the vector-treated bleb area with either the canine or human BEST1 transgenes (FIGS. 3C and 3D, Lower panel and FIGS. 10 and 11). No differences in the clinical picture or response to the AAV-BEST1 treatment were observed between genders.

Retinas were preserved after AAV-hBEST1 treatment in three cBest models [cmr1 (R25*/R25*), cmr1/cmr3 (R25*/P463fs), and cmr3 (P463fs/P463fs)] in comparison with the wild-type control and cBest untreated eyes. cBest eyes were injected with AAV-hBEST1 (2×10¹¹ vg/mL) at 27 wk (cmr1), 45 wk (cmr1/cmr3), or 63 wk (cmr3) of age, and evaluated by IHC at 103 wk, 51 wk, or 207 wk p.i., respectively (FIG. 10). No apparent abnormalities within the treated areas were detected up to 207 wk p.i. Note the RPE apical extensions projecting into subretinal space in all treated eyes (EZRIN). The untreated cBest control (Far Right panel) shows lack of RPE apical microvilli, RPE hypertrophy (EZRIN, RPE65), and accumulation of lipofuscin granules within the RPE monolayer along with autofluorescent deposits in the subretinal space.

Representative confocal photomicrographs are shown in FIG. 11A depicting a cBest (R25*/P463fs) retina 79 weeks post AAV-hBEST1 injection (2.5×10¹¹ vg/mL) and double-labeled with BEST1 (RPE, darker color) and SLC16A1 (RPE, lighter color). A cross-sectional overview from the surgical bleb area (FIG. 11B), through the adjacent penumbral region (FIG. 11C), and toward the contiguous extent outside of the injection zone (FIG. 11D) is shown in FIGS. 11B-11D. A direct correlation between the degree of restoration of the RPE-PR interface structure and BEST1 transgene expression was observed as highlighted in the magnified images. A remarkable extension of RPE apical projections within the treated region with augmented BEST1 was observed (FIG. 11B); presence of vestigial microvilli [c-MV (lighter arrowheads) and rod-MV (darker arrowheads)] in the bleb penumbra associated with patchy distribution of BEST1 (weak red signals within individual RPE cells) and RPE-PR microdetachment (FIG. 11C); formation of subretinal lesions in the absence of both BEST1 expression and RPE apical processes outside of the treatment zone (FIG. 11D). Scalloped and unelaborated RPE apical surface and massive intracellular deposits appeared as granular aggregates within cBest mutant RPE (FIG. 11A, upper panel, Non-injected; FIG. 11D, close-up). In the zone of detachment, cellular debris creeping into subretinal space (asterisks) corresponds to the Muller glia, and reflect retinal remodeling in response to stress. [Scale bars, 100 μm (Upper) and 10 μm (FIGS. 11A-11D).]

Correction of Light-Modulated Microdetachments with Gene Therapy.

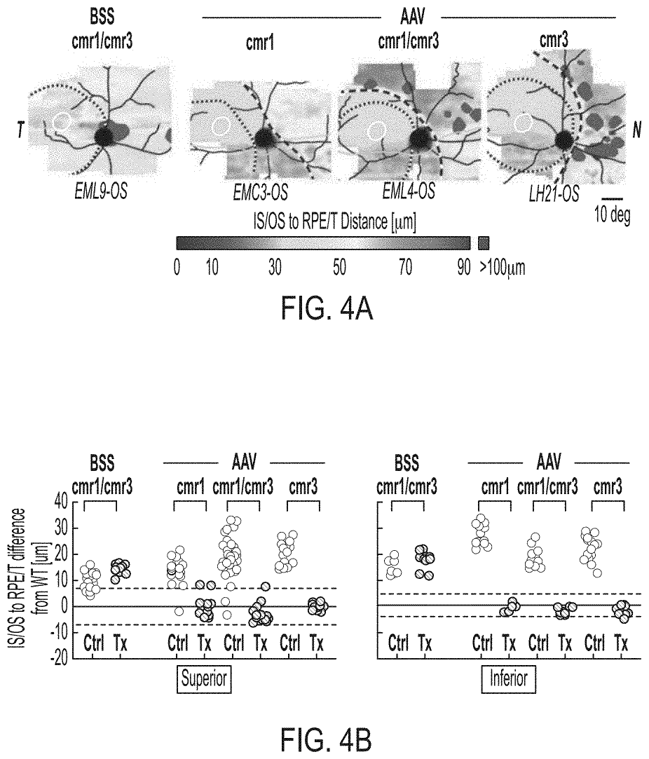

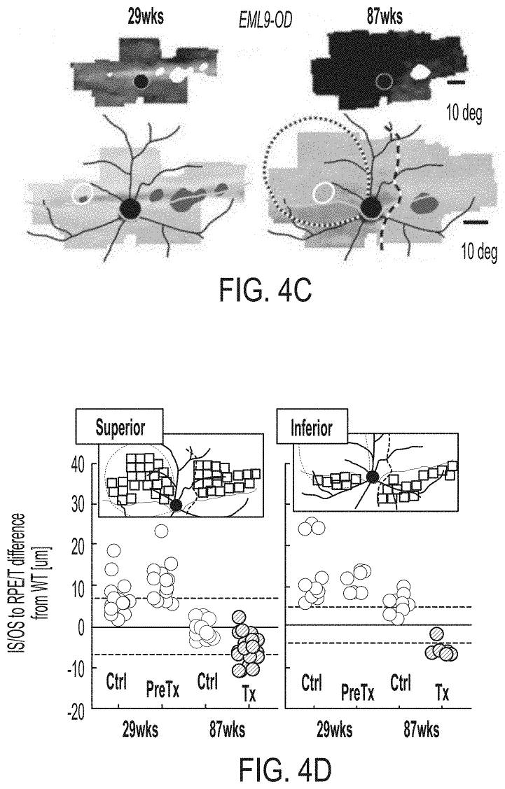

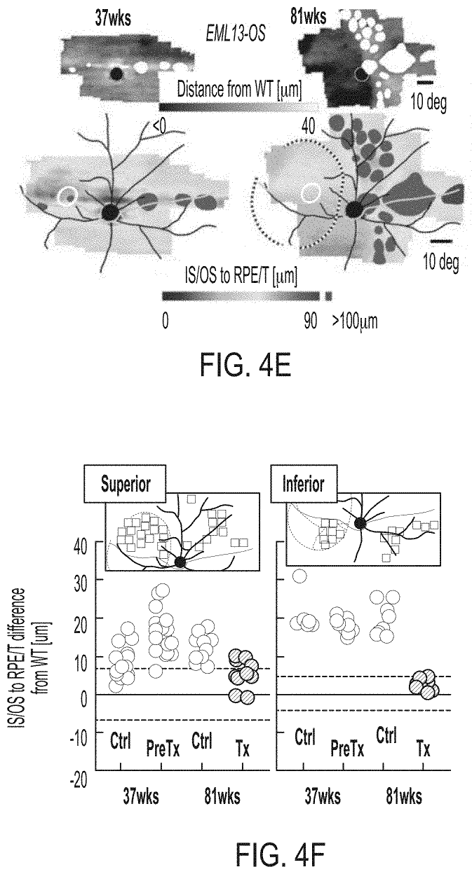

To understand the consequences of BEST1 gene augmentation therapy on retinal regions without ophthalmoscopically detectable retinal detachments, IS/OS-RPE/T distance was measured topographically within and outside subretinal blebs, A representative result with a control subretinal BSS injection at age 69 wk in a cBest (R25*/P463fs) dog showed homogeneous microdetachment covering all the imaged retina at age 87 wk (FIG. 4A). The average microdetachment extent (IS/OS-RPE/T distance of the BSS-injected mutant dog subtracted from colocalized measurements performed in WT eyes) was 11.6 μm in the superior retina and 16.7 μm in the inferior retina (FIG. 4B), consistent with uninjected cBest eyes. Subretinal AAV gene therapy, on the other hand, resulted in substantial reduction of the IS/OS-RPE/T distance in treated regions. EMC3-OS, EML4-OS, and LH21-OS demonstrate results in three genotypes treated with gene therapy using the human BEST1 transgene with titers of ˜2×10¹¹ vg/mL (FIG. 4A and Table 1). In each case, there was significant reduction of the IS/OS-RPE/T distance in the treated bleb. Notably, gross retinal detachments (darker color) were only detectable outside the treatment region (FIG. 4A). Quantitative measurements showed complete amelioration of the microdetachments, with the IS/OS-RPE/T distance returning to WT levels both in superior and inferior retinal regions treated with subretinal gene therapy (FIG. 4B, filled symbols) but not in retinal regions away from the treatment bleb (FIG. 4B, unfilled symbols).

The region of efficacy with subretinal gene therapy is often shown to extend beyond the bleb formed at the time of the surgery to include a penumbral region. In cBest dogs with successful gene therapy, there was also a penumbral region but it appeared to be qualitatively larger than typically encountered previously (FIG. 4A). In some of the most extreme examples, pretreatment maps of retina-wide microdetachment were found to be necessary to demonstrate the extent of penumbral expansion. EML9-OD, for example, at age 29 wk showed a retina-wide microdetachment that was most extreme along the visual streak and included several regions with gross retinal detachments (FIG. 4C). Gene therapy was performed at 69 wk. At 87 wk, microdetachments across the whole retina imaged as well as the majority of the gross retinal detachments had disappeared (FIG. 4C), and quantitative measures showed a normal or thinner IS/OS-RPE/T distance in superior and inferior retinal locations (FIG. 4D). Importantly, IS/OS-RPE/T distance showed substantial improvements at retinal locations corresponding to the bleb formed at the time of the injection as well as in nasal retinal control regions in the same eye. This extreme example of penumbral expansion is likely explained by greater diffusion of the vector via the microdetachment in cBest eyes that resulted in the transduction of the RPE at sites substantially more distant than the initial bleb. A more typical example with a delimited penumbral expansion is illustrated for comparison. EML13-OS at age 37 wk showed microdetachments retina-wide that were especially prominent in the temporal retina and along the visual streak; there were also several gross retinal detachments along the visual streak (FIG. 4E). Gene therapy was performed at 45 wk. At 81 wk of age, both superior and inferior retina temporal to the optic nerve was lacking micro- and macrodetachments, whereas the untreated nasal retina had retained the microdetachments as well as formed a large number of macrodetachments (FIG. 4E). Quantitative results confirmed the treatment effect (FIG. 4F), which did not reach the nasal retina, unlike EML9-OD.

To understand the potential consequences of gene therapy on retinal degeneration, ONL thickness was mapped across treated eyes (FIG. 8B). Treated retinal regions showing disappearance of microdetachments tended to also correspond to normal ONL thickness, whereas untreated regions retaining microdetachments tended to show hyperthick or normal, or in some regions, thinned ONL (FIG. 8B). In summary, AAV-mediated gene augmentation therapy in canine bestrophinopathies appears to promote a sustained reversal of gross retinal detachments, reestablishment of a close contact between RPE and PRs, and return of ONL thickness to normal values.

### Human Autosomal Recessive Bestrophinopathies: Structure and Function.

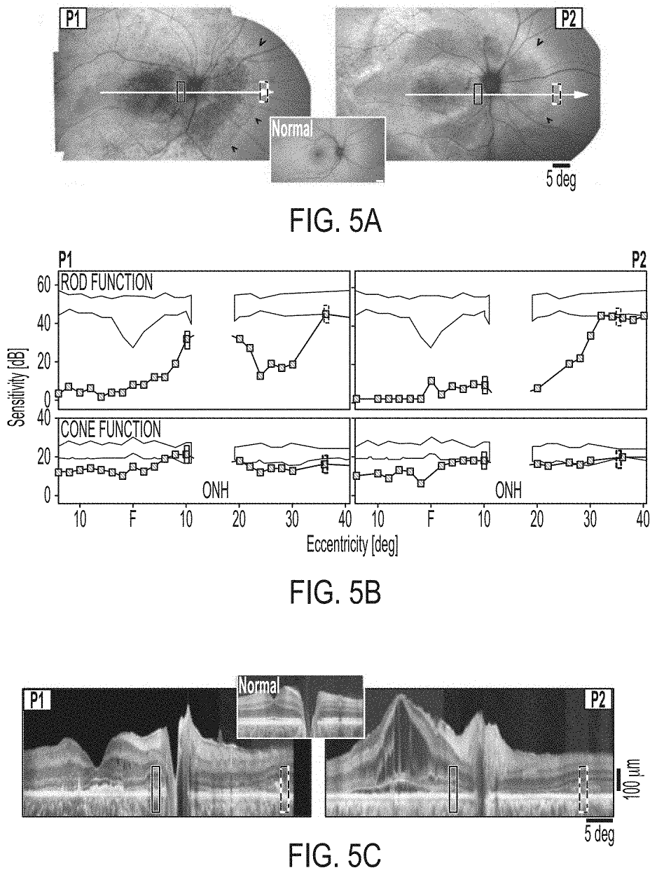

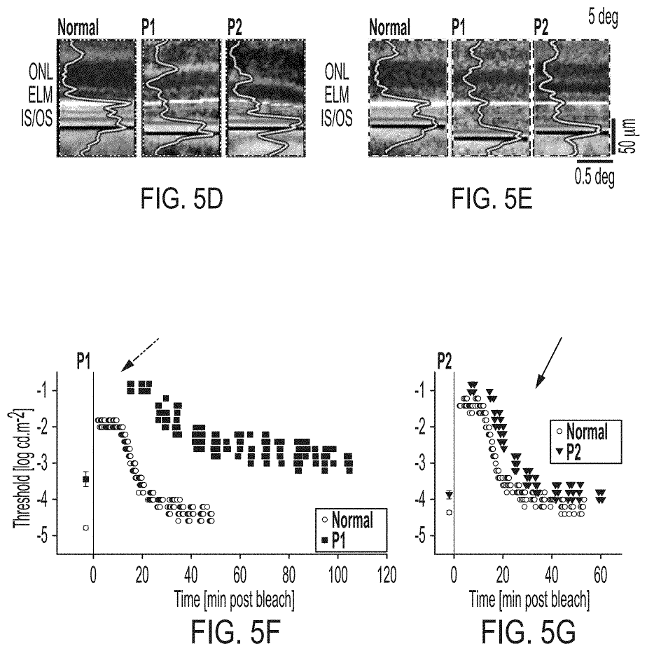

To facilitate clinical translation of successful gene therapy in BEST1-mutant dogs, studies were performed to better understand the human pathophysiology of autosomal recessive bestrophinopathies (ARBs) and to gain insight into the distribution of retina-wide disease beyond the gross ophthalmoscopically detectable lesions previously described. Data are shown (FIGS. 5A-5G) from two patients: P1 was a 39-y-old woman with a best corrected visual acuity of 20/100 carrying biallelic BEST1 mutations (c.341T>C/c.400C>G), whereas P2 was a 36-y-old man with 20/60 acuity also carrying biallelic mutations (c.95T>C/c.102C>T) in BEST1. In both patients, mutant alleles segregated with clinically unaffected parents. Ultrawide imaging of RPE health taking advantage of the natural autofluorescence of lipofuscin granules they contain showed widespread and extensive abnormalities consisting of regions of relative hyper- or hypoautofluorescence and local heterogeneity. Of note, there was a distinct transition zone (FIG. 5A, arrowheads) in the nasal midperipheral retina demarcating the healthier nasal peripheral retina.

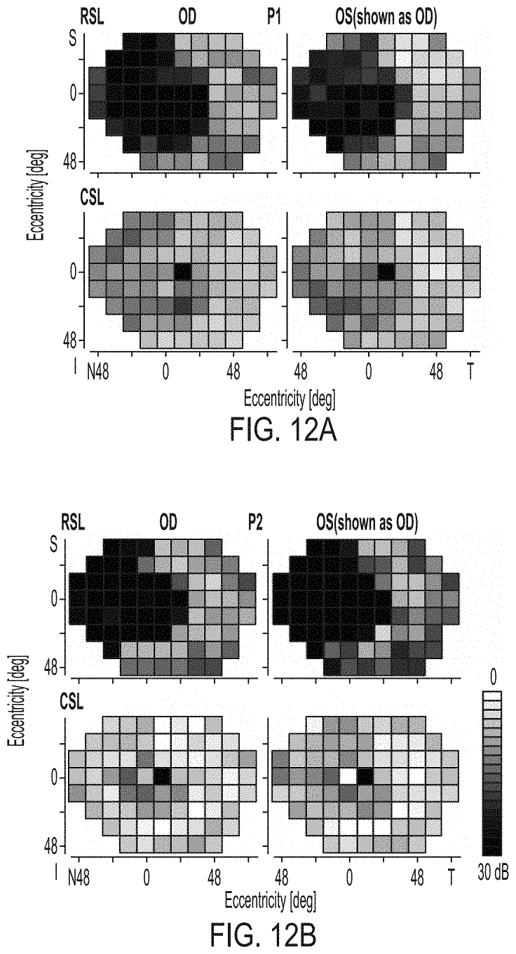

Rod and cone function was sampled at high density along the horizontal meridian to better understand the topography of vision loss and its correspondence to retinal structural abnormalities. Both patients demonstrated a deep (>3 log) loss of rod-mediated sensitivity centrally in long-term dark-adapted eyes; there was relative preservation of rod function in the temporal field (nasal retina) in both patients and the parapapillary area in one patient (FIG. 5B, Upper panel). Surprisingly, cone-mediated function in light-adapted eyes demonstrated only a moderate loss (<1 log) or normal or near-normal results (FIG. 5B, Lower panel). Rod and cone function sampled across the full extent of the visual field corroborated and extended these findings and showed strong interocular symmetry (FIGS. 12A and 12B). Rod (RSL) and cone sensitivity loss (CSL) maps of both eyes of two patients with ARB. Large and symmetric central areas of severe RSL were surrounded by relatively retained function in the temporal field. Cone function is relatively less affected and CSL is relatively uniform across the visual fields. Physiological blind spot is shown as black square at 12° in the temporal field.

Cross-sectional imaging with OCT was performed to evaluate the retinal lamination abnormalities along the horizontal meridian crossing the fovea (FIG. 5C). There was not a consistent light-exposure history at the time of OCT imaging. Both patients showed a significant loss of ONL and abnormalities at the level of photoreceptor IS/OS in the outer retina across most of the central retina. P2, in addition, showed intraretinal cystic spaces and the detachment of the central retina from the RPE, likely due to accumulation of subretinal fluid. The retinal lamination showed relative normalization in the parapapillary region (FIG. 5C, dark-colored rectangles) and beyond the nasal midperipheral transition (FIG. 5C, light-colored rectangles). Analyses of the two regions of interest showed detectable but abnormally thinned ONL, and detectable IS/OS and cone outer segment tip (COST) with low peak signal in both patients (FIGS. 5D and 5E). In P1, the distances from the ELM to IS/OS and IS/OS to COST were comparable to normal. There appeared to be a hyposcattering layer distal to COST, and the RPE appeared hyperthick (FIG. 5D, Middle panel). In P2, ELM to IS/OS appeared shorter than normal, whereas IS/OS-to-COST distance was comparable to normal. COST-to-ROST/RPE distance appeared greater than normal with an intervening indistinct hyposcattering layer; the RPE appeared to be comparable in thickness to normal (FIG. 5D, Right panel). Analysis of the outer retina in the nasal midperipheral region in P1 showed ELM-to-IS/OS, IS/OS-to-COST, and COST-to-ROST/RPE distances greater than normal and RPE thickness which was comparable to normal (FIG. 5E, Middle panel). P2 features appeared intermediate between P1 and normal (FIG. 5E, Right panel).

To understand the implications of structural abnormalities at the level of the outer retina and RPE for the kinetics of retinoid transfer between these cellular layers, dark-adaptation testing was performed. At the parapapillary location shown in FIG. 5D, dark-adapted thresholds of P1 were rod-mediated but 1.3 log unit-elevated (FIG. 5F). By 22.5 min following a light exposure, the P1 results had remained cone-mediated on a plateau whereas normal was already within 1 log unit of the final dark-adapted threshold. By 50 min, P1 rod results were still 1 log-elevated whereas normal recovery was complete (FIG. 5F). At the midperipheral nasal retinal location shown in FIG. 5E, dark-adapted thresholds of P2 were rod-mediated and −0.5 log unit-elevated compared with normal (FIG. 5G). By 14.5 min following a light exposure, there was first evidence of rod function which was only incrementally slower than the 11-min cone-rod break in normal. The rate of rod recovery was similar to normal (FIG. 5G). In summary, rod dark-adaptation kinetics of P1 at the parapapillary locus showed an extremely slow time course, whereas dark-adaptation kinetics of rod function of P2 at the midperipheral locus was closer to normal (FIGS. 5F and 5G).

The RPE has a key role in maintaining the metabolically active environment of the subretinal space. Due to the dynamic relationship with adjacent retinal layers, mutations in RPE-specific genes often adversely affect the neighboring sensory neurons, leading to loss of visual function and PR degeneration. Mutations in BEST1 are known to disrupt transepithelial ion and fluid transport in response to abnormal levels of intracellular calcium. Abnormal RPE calcium signaling is also thought to lead to dysregulation of other pathways through altered expression and interactions of Ca²⁺-sensitive proteins. Based on findings in cBest, one such protein is EZRIN, a membrane-cytoskeleton linker essential for the formation and proper maturation of RPE apical MV. It has been demonstrated that the activation of EZRIN's membrane-F-actin cross-linking function occurs directly in response to Ca²⁺ transients, and Ezrin-KO mice exhibit a substantial decrease in elaboration of RPE MV. The apparent underdevelopment of RPE apical MV found in the BEST1-mutant RPE is consistent with these findings. Furthermore, comparative IHC assessments with other IRD models demonstrated that these major structural alterations associated with microvillar ensheathment are specific to the primary RPE channelopathy triggered by BEST1 mutations, and not secondary to cone dysfunction and degeneration.

The structural components of RPE apical processes are very different from those of nonmotile intestinal microvilli. The presence of contractile proteins (such as myosin) in the RPE apical microvilli, and also molecules typically found at the sites of cell attachments, suggests that the RPE actively adheres to, and exerts tension on, the neural retina. The dearth of a proper microvillar ensheathment at the RPE-PR interface in cBest, and thus an absence of physical and electrostatic support by these projections to the PR OS, would be expected to weaken the adhesive forces and lead to separation of the RPE-PR complex retina-wide. The microdetachment of the PR layer from the underlying RPE found in cBest at the earliest stages of disease would be consistent with this process. Moreover, the presence of contractile elements in the RPE apical projections and the fact that they have evolved from cells in which pigment migration occurred indicate that MV are capable of active contraction while interdigitating with PR OS, and are destined to facilitate circadian phagocytic activity. A single RPE cell can accommodate about 30 to 50 PRs, depending on the retinal location and packing density; the elaborate network of microvilli allows each RPE cell to handle such a high metabolic load on a daily basis. Insights from proteomic profiling support this argument. There is an enriched fraction of retinoid-processing proteins expressed along the RPE apical MV, together with a number of channel proteins and transporters (e.g., Na⁺/K⁺ ATPase) central to the efficient transport of water, ions, and metabolites between the RPE and PR OS. Considering the topographic differences in the size of RPE cells and taking into account the density and length of MV quantified in this study, MV extensions expand the functional surface of a single RPE cell by 20- to 30-fold in the central retina, which is consistent with earlier estimates. This number is even higher (˜50-fold) for the small RPE cells in the macular region that adapted to a higher turnover rate of shed POS while facing the most densely packed PRs. Such dramatic reduction in a total apical surface area in BEST1-mutant RPE will lead to a chronic delay in processing of metabolites, and arrest the abilities of the RPE to maintain both the proper cell volume as well as chemical composition and physiological pH levels in the subretinal space. Since these factors are essential for retinal adhesion, any limitation in the RPE transporting system will alter the balance of hydrostatic forces and result in decreased osmoelastic properties of the RPE-PR complex with subsequent separation from the neuroretina. Indeed, the primary serous detachment in human and canine bestrophinopathy is first evident in the fovea, the central area of highest metabolic activity. The absence of high-reaching RPE apical processes, which in the structurally intact retina tightly wrap COS up to the ellipsoids, would explain the predilection of this cone-rich structure for its primary detachment in bestrophinopathies. There would be almost exclusive reliance on the frictional interactions with the MV. This is consistent with observations in cBest, documenting formation of the focal previtelliform lesions within the canine fovea-like area of the area centralis, and also the susceptibility of other central cone-rich areas (like visual streak) to subretinal detachment.

The major expansion of microdetachments in cBest upon exposure to dim and moderate light intensities was an unexpected result. In normal eyes, light exposure is known to change molecular composition of the subretinal space. There is also evidence that measurable structural changes occur in the normal outer retina with light exposure, such as changes in outer segment length, hydration of the subretinal space, increased actin staining along RPE apical MV, and phototropism of outer segments. However, all of the normal changes are substantially smaller than those measured in cBest. For example, normal human eyes showed changes of ˜1 μm, and normal mouse eyes showed changes of ˜4 μm in the outer retina, compared with ˜18-μm expansion of the subretinal space driven by light in cBest. Human ARB has only recently been recognized and the literature on the earliest disease stages is limited. The recessive cBest disease appears to have phenotypic similarities to both dominant and recessive bestrophinopathies in humans. In patients with Best disease (BVMD), there has been some controversy regarding the structural features of retinal regions surrounding vitelliform or later-stage lesions, or retinas in the previtelliform stage of disease. Some studies have demonstrated minor abnormalities at the level of the RPE-PR interface, whereas results from others support no detectable structural defects. Contributing to this controversy could be genotype, the resolution of different methodological approaches used, or light history preceding the imaging. Indeed, light-dependent outer retinal changes have been described in BVMD using methods such as those disclosed herein; still, the magnitude of the changes in patients was smaller (˜2 μm) than in cBest. In general, however, the abnormal response of the affected retina to light stimuli could be related to the markedly reduced light peak/dark trough ratio in the electrooculogram, a finding consistent in all, even presymptomatic, Best Disease patients.

Of importance, both the micro- and macrodetachments in cBest had adverse effects on photoreceptor health: Regions of microdetachment tended to correspond to hyperthick ONL, whereas large lesions with gross macrodetachment showed thinning of ONL. Smaller lesions with macrodetachment could not be assessed with the sampling methods used here. ONL contains the nuclei of all rods and cones, and classic studies in animal models and human eye donors have generally shown thinning of the ONL with disease progression. Less well known are some of the earliest stages of retinal disease showing ONL thickening, which has only become measurable with the advancement of in vivo imaging methods. Human studies have previously demonstrated such ONL thickening in early stages of retinal diseases. There has also been evidence in animal studies of ONL thickening associated with retinal stress. The hyperthick regions of ONL mapped in cBest when examined microscopically showed the number of PR nuclei to be comparable to control, suggesting a greater internuclear spacing within the ONL, likely corresponding to a level of retinal stress that is below the apoptosis threshold. Gross retinal detachments, on the other hand, may cause greater retinal stress and progressive degeneration.

To prevent the photoreceptor and vision loss associated with BEST1 mutations, subretinal gene augmentation therapy directed to retinal areas with macro- and microdetachments was performed. Results showed that AAV-mediated BEST1 gene augmentation is safe, reverses the clinically obvious lesions, ameliorates the diffuse microdetachments, and results in normalization of hyperthick ONL. Furthermore, gene therapy was successful in three distinct BEST1 genotypes with both focal and multifocal presentations, and confirmed long-term durability of the treatment effect. At the molecular level, the ability of the canine as well as the human BEST1 transgene to correct the apposition of the RPE-PR complex and restore the cytoarchitecture of this critical interface was confirmed. This study suggests that early as well as more advanced stages of autosomal recessive disease are sensible to approach with this therapy. Further studies utilizing human inducible pluripotent stem cell (hiPSC)-derived RPE models derived from patients harboring autosomal Best1 mutations will determine whether the gene augmentation approach would also be beneficial for BVMD patients.

To facilitate the clinical translation of successful gene augmentation therapy, ARB patients were studied to gain insight into their retina-wide disease. Consistent with most, but not all, previous descriptions, retinal disease in ARB patients extended well beyond the macula into the midperiphery. Retinotopic mapping of en face and cross-sectional imaging and rod and cone function demonstrated the existence of a distinct transition from disease to health in the midperipheral retina, a feature not previously emphasized. Within the diseased region, severe abnormalities in retinal structure were associated with severe loss of rod function; unexpectedly, cone function was relatively retained. Rod dysfunction within the central retina was also associated with extreme slowing of the retinoid cycle, whereas the healthier periphery showed near-normal recycling of the retinoids. There are at least two retinoid cycles that provide the 11-cis-retinal chromophore to photoreceptor pigments. The canonical retinoid cycle functions in the RPE to produce chromophore for rod and cone PRs. The retinal retinoid cycle, on the other hand, is thought to regenerate chromophore within the retina for the specific use of cones. The abnormal RPE-PR interface in Best disease would most likely affect the chromophore delivery from the canonical RPE retinoid cycle; the retinal retinoid cycle may be relatively unaffected, thus explaining the greater retention of cone function.

In summary, as disclosed herein, new molecular contributors to the pathophysiology of bestrophinopathies at the RPE-PR interface were surprisingly uncovered. The earliest expression of disease was discovered—a diffuse microdetachment potentiated by light exposure that was easily detectable by in vivo imaging. AAV-mediated BEST1 augmentation gene therapy reversed both the grossly obvious lesions and microdetachments, and restored the cytoarchitecture of the RPE-PR interface. Evaluation of ARB patients showed retinotopic distribution and properties of structural and functional defects beyond that expected from PR degeneration. Such visual dysfunction may be expected to improve upon successful application of BEST1 gene augmentation therapy to patients affected with bestrophinopathies.

### Example 2

The vector technology of Example 2 was designed to suppress the expression of endogenous BEST1 mRNA (both the mutated and the normal copy) using RNA interference. These vectors simultaneously replace the endogenous BEST1 mRNA with normal BEST1 mRNA to produce only normal protein. The technology uses adeno-associated virus to deliver an intronless copy of the BEST1 gene plus a gene for a small hairpin RNA (shRNA) that leads to the production of a small interfering RNA (siRNA). The BEST1 gene has been rendered resistant to the siRNA because of silent mutations in its reading frame. Two shRNAs, and therefore two modified human BEST1 genes, were designed. Both BEST1 genes are driven by a 623 bp fragment of the human VMD2 promoter. The BEST1 cDNA is preceded by a synthetic intron and followed by a poly adenylation sequence, both derived from the SV40 virus. In one case, shRNA05 is driven by the RNA polymerase III (pol III) H1 promoter, and in the other, shRNA744 it is driven by the pol III U6 promoter. A sequence of six thymidines, serves as a termination sequence for each shRNA. To identify these active shRNAs, nine potential siRNA or shRNA sequences were screened.

The genetic sequences encoding the shRNAs are as follows:

shRNA05
(SEQ ID NO: 1)

| | |
|---|---|
| CGUCAAAGCUUCACAGUGU | UUCAAGAGA |

ACACUGUGAAGCUUUGACG

| | | |
|---|---|---|
| shRNA05 | | shRNA05 |
| sense | Loop | anti-sense |
| (SEQ ID NO: 2) | (SEQ ID NO: 7) | (SEQ ID NO: 3) |

shRNA744
(SEQ ID NO: 4)
AAGAACUCGCCAUAUAGCAGC CUCGAG
GCUGCUAUAUGGCGAGUUCUU

| | | |
|---|---|---|
| shRNA744 antisense | Loop | shRNA744 sense |
| (SEQ ID NO: 5) | (SEQ ID NO: 8) | (SEQ ID NO: 6) |

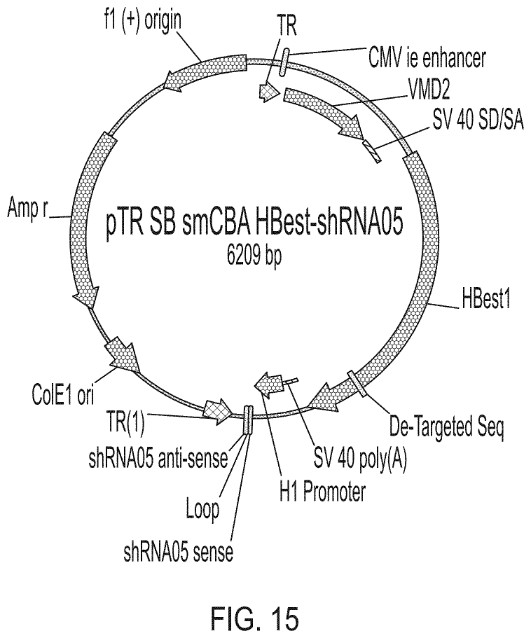

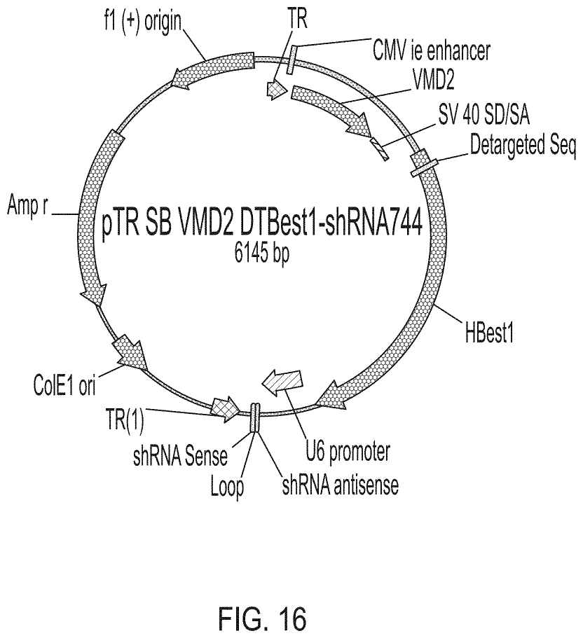

Maps of exemplary AAV vectors comprising heterologous nucleic acids encoding shRNA05 and shRNA744 as well as a hBEST1 gene that includes a de-targeted sequence (e.g., one of SEQ ID NOs: 10 or 11), which are used to produce the disclosed rAAV particles, are shown in FIGS. 15 and 16, respectively. Both sequences are driven by a VMD2 promoter.

In some embodiments, disclosure provides an shRNA05 sense strand that comprises a sense strand comprising the nucleotide sequence of SEQ ID NO: 2, plus an additional nucleotide immediately prior to the first cytosine of this sequence. In certain embodiments, this additional nucleotide comprises a cytosine (C).

In some embodiments, the disclosure provides an shRNA05 that comprises an antisense strand comprising the nucleotide sequence of SEQ ID NO: 3.

An exemplary genetic sequence corresponding to the region of the vector encoding the pol III H1 promoter, shRNA05, and termination sequence is as follows:

(SEQ ID NO: 20)
TAAAACGACGGCCAGTGAATTCATATTTGCATGTC
GCTATGTGTTCTGGGAAATCACCATAAACGTGAAA
TGTCTTTGGATTTGGGAATCTTATAAGTTCTGTAT
GAGACCACTcggatccCGTCAAAGCTTCACAGTGT
TTCAAGAGAACACTGTGAAGCTTTGACGTTTTTT.

This sequence further includes a BamHI endonuclease site (ggatcc) to facilitate screening and ensure that the start site of the shRNA05 would be positioned 25 nucleotides downstream of the H1 promoter TATA box (TATAA). Accordingly, in some embodiments, an shRNA (e.g., shRNA05) encoded by a nucleic acid comprising this sequence (and/or the complement thereof) is transcribed in a host cell (e.g., in a subject, for example in a human subject, treated with the vector). In some embodiments, two or more different shRNAs (e.g., having different start sites and/or termination sites, for example differing from shRNA05 by one or two additional or fewer nucleotides) are transcribed in a host cell.

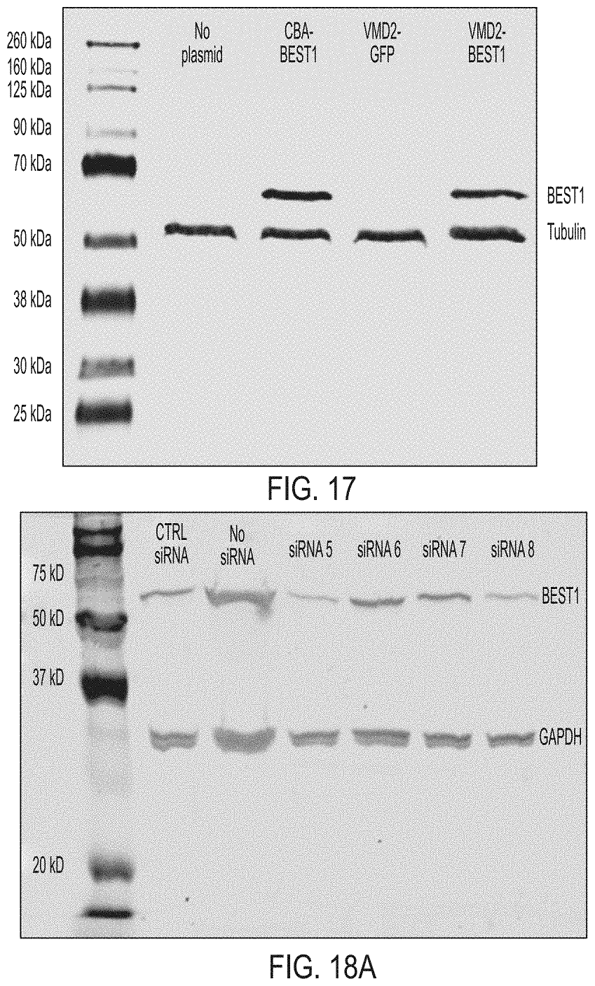

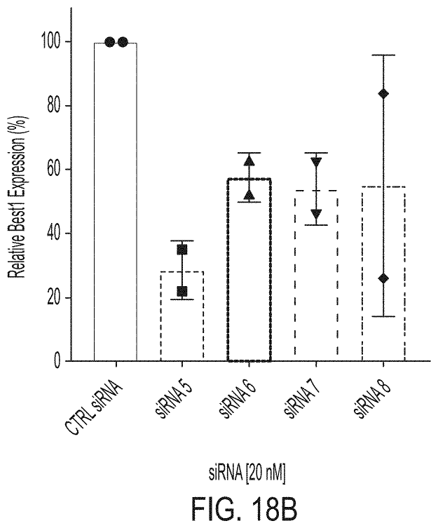

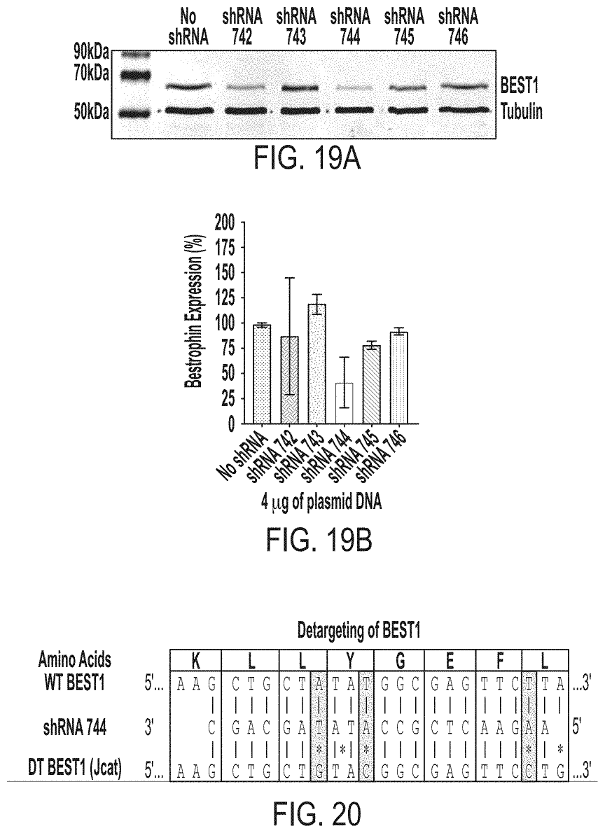

FIG. 17 shows that the VMD2 promoter works well in cell culture. HEK293T cells were transfected with plasmids expressing GFP or Best1 using the Chicken beta actin promoter (CBA) or the VMD2 promoter. Protein lysates were separated on polyacrylamide gels and expression of bestrophin (Best1) was detected by Western Blot and normalized to the expression of beta-tubulin to show even loading of the gel. FIGS. 18A and 18B shows that Best1-specific siRNA is functional. Transfection of HEK293T stably expressing BEST1 led to a 75% reduction in Bestrophin (Best1) protein. 20 nM siRNA was employed can cells were analyzed 48 hours after transfection. Western blot (FIG. 18A), Knock-down of BEST1 was compared by standardization of band intensity between Best1 and Tubulin (Best1/Tubulin) (FIG. 18B). FIGS. 19A and 19B show Best1 shRNA is active: HEK293T-BEST1 cells were transfected with 4 μg of the indicated plasmid. Cells were harvested 48 hrs after transfection. Expression of BEST1 was determined by Western Blot (FIG. 19A). Knock-down of BEST1 was compared by standardization of band intensity between Best1 and Tubulin (Best1/Tubulin) (FIG. 19B). FIG. 20 shows de-targeting of Best1. Silent mutations (base changes in the third position of codons) were used to remove an siRNA target site from Best1 mRNA. The example disclosed is for shRNA744. SEQ ID NOs: 15-17 correspond to the sequences from top to bottom.

### Materials and Methods

### Canine BEST1 Models and In Vivo Retinal Imaging.

cBest-mutant dogs (n=18) of both sexes (12 M and 6 F) harboring either homozygous (c.73C>T) (p.R25*/R25*) or (c.1388delC) (p.P463fs/P463fs) or biallelic (c.73C>T/1388delC) (p.R25*/P463fs) mutations in cBEST1 (GB*NM 001097545) were included. For ease of annotating the multipanel figures, the three genotypes, respectively, are referred to as cmr1, cmr3, and cmr1/cmr3. The study was conducted in comparison with control cross-bred dogs (n=12; 7 M and 5 F) (Table 1). All animals were bred and maintained at the Retinal Disease Studies Facility (RDSF). The studies were carried out in strict accordance with the recommendations in the Guide for the Care and Use of Laboratory Animals of the NIH and in compliance with the Association for Research in Vision and Ophthalmology Statement for the Use of Animals in Ophthalmic and Vision Research. The protocols were approved by the Institutional Animal Care and Use Committee of the University of Pennsylvania (IACUC nos. 804956 and 803422). En face and retinal cross-sectional imaging was performed with the dogs under general anesthesia as previously described.

### Human Subjects.

Light-adapted and two-color dark-adapted function was measured at 2° intervals across the central visual field (central 60° along horizontal and vertical meridians) and at 12° intervals throughout the visual field. Photoreceptor mediation under dark-adapted conditions was determined by the sensitivity difference between 500- and 650-nm stimuli. Dark-adaptation kinetics was evaluated similar to techniques previously described (92-94) using an LED-based dark adaptometer (Roland Consult) and a short-duration (30 s) moderate light exposure from a clinical short-wavelength autofluorescence imaging device (25% laser output; Spectralis HRA; Heidelberg Engineering). Optical coherence tomography (OCT) was used to analyze laminar architecture across the retina. Retinal cross-sections were recorded with a spectral-domain (SD) OCT system (RTVue-100; Optovue). Postacquisition data analysis was performed with custom programs (MATLAB 7.5; MathWorks). Recording and analysis techniques have been previously described (30, 31, 94). Longitudinal reflectivity profiles (LRPs) were used to identify retinal features. A confocal scanning laser ophthalmoscope (Spectralis HRA; Heidelberg Engineering) was used to record en face images and estimate RPE health with short-wavelength reduced-illuminance autofluorescence imaging (SW-RAFI) as previously described (95). All images were acquired with the high-speed mode (30°×30° square field or 50° circular field).

### Canine BEST1 Models and in Vivo Retinal Imaging.

Overlapping en face images of reflectivity with near-infrared illumination (820 nm) were obtained (Spectralis HRA+OCT) with 30°- and 55°-diameter lenses to delineate fundus features such as the optic nerve, retinal blood vessels, boundaries of injection blebs, retinotomy sites, and other local changes. Custom programs (MATLAB 7.5; MathWorks) were used to digitally stitch individual photos into a retina-wide panorama. Short-wavelength autofluorescence and reflectance imaging was used to outline the boundary of the tapetum and pigmented RPE. Spectraldomain optical coherence tomography (SD-OCT) was performed with overlapping (30°×25°) raster scans across large regions of the retina. Postacquisition processing of OCT data was performed with custom programs (MATLAB 7.5). For retina-wide topographic analysis, integrated backscatter intensity of each raster scan was used to position its precise location and orientation relative to the retinal features visible on the retinawide mosaic formed by near-infrared reflectance (NIR) images. Individual LRPs forming all registered raster scans were allotted to regularly spaced bins (1°×1°) in a rectangular coordinate system centered at the optic nerve; LRPs in each bin were aligned and averaged. Intraretinal peaks and boundaries corresponding to the OPL, ELM, IS/OS, and RPE/T were segmented using both intensity and slope information of backscatter signal along each LRP. Topographic maps of ONL thickness were generated from the OPL-to-ELM distance, and maps of IS/OSto-RPE/T thickness were generated from the distance between these peaks. For all topographic results, locations of blood vessels, optic nerve head, bleb, tapetum boundaries, and fovea-like area (24) were overlaid for reference. First, maps from WT dogs were registered by the centers of the optic nerve head and rotated to bring the fovea-like areas in congruence, and a map of mean WT topography was derived. The fovea-like area of cBest mutant dogs was determined by superimposing a WT template onto mutant eyes by alignment of the optic nerve head, major superior blood vessels, and boundary of the tapetum. Next, cBEST1-mutant maps were registered to the WT map by the center of the optic nerve and estimated fovea-like area, and difference maps were derived. Difference maps were sampled within and outside treatment blebs for each eye. The relation between exposure to light and changes to outer retinal structure was evaluated by two approaches. In a subset of eyes, cross-sectional OCT imaging was performed early in each experimental session followed initially by autofluorescence imaging with a bright short-wavelength light followed subsequently by further OCT imaging. OCT records obtained early in such sessions were considered to be from retinas exposed to less light compared with records obtained late, although the exact light exposure could not be quantified. In another subset of eight eyes, OCT recordings were performed after overnight dark adaptation, and serially in a dark room following short intervals of short-wavelength light exposure from a cSLO. In three of the eyes, five increasingly greater light exposures were used: L1: laser, 20%; duration, 60 s; L2: laser, 25%; duration, 30 s; L3: laser, 50%; duration, 30 s; L4: laser, 100%; duration, 30 s; L5: laser, 100%; duration, 300 s. In three eyes, only L4 and L5 were used. In two additional eyes, only L5 was used to follow the recovery of light-mediated microdetachment over a 24-h period. The standard (100%) laser setting is estimated to correspond to a human retinal irradiance of 330 μW·cm-2 at 488-nm wavelength (98). In both approaches, the areas selected for analysis were based on near-infrared imaging of the fundus with the cSLO before the start of the study, and excluded areas where overt clinically visible macrodetachments were located.

### Subretinal Injections and Postoperative Procedures.

Subretinal injections of recombinant AAV2/2 delivering either the cBEST1 or hBEST1 transgene under control of the human VMD2 promoter (46) were performed under general anesthesia following previously published procedures (46, 82, 97). Vector production and validation have been detailed previously (46). Injection volumes ranging from 50 to 180 μL of the viral vector solution (titer range of 0.1 to 5×10¹¹ vg/mL) (Table 1) were delivered subretinally using a custom-modified RetinaJect subretinal injector (SurModics) (97) under direct visualization with an operating microscope via a transvitreal approach without vitrectomy. An anterior chamber paracentesis was performed immediately after injection to prevent increase in intraocular pressure. Directly after injection, formation of a subretinal bleb was documented by fundus photography (RetCam Shuttle; Clarity Medical Systems). In all cases, the surgical bleb flattened and the retina reattached within 24 to 48 h p.i. Ophthalmic examinations, including biomicroscopy, indirect ophthalmoscopy, and fundus photography, were conducted on a regular basis (24 h, 48 h, and 5 d p.i., and then weekly for the first 2 mo followed by a monthly eye examination thereafter) throughout the injection-end point evaluation time interval. Postoperative management was performed as described previously (46).

### Histological and Immunohistochemical Evaluations.

Ocular tissues for ex vivo analyses were collected as previously described (24, 99). All efforts were made to improve animal welfare and minimize discomfort. For all ex vivo assessments, cBest and control (WT) eyes were fixed in 4% paraformaldehyde, embedded in optimal cutting temperature media, and processed as reported previously (99). Histological assessments were made using standard hematoxylin/eosin (H&E) staining, and all immunohistochemical experiments were performed on 10-μm-thick cryosections following established protocols (46, 99). Briefly, retinal cryosections were permeabilized with 1×PBS/0.25% Triton X-100, blocked for 1 h at room temperature, and incubated overnight with a primary antibody (Table 2). For multicolor labeling, primary antibodies were combined with Alexa Fluor 488 phalloidin (Thermo Fisher Scientific) or PNA-AF647 (L32460; Molecular Probes), followed by incubation with a corresponding secondary antibody (Alexa Fluor) for 1 h. The slides were examined by epifluorescence or transmitted light microscopy (Axioplan; Carl Zeiss Meditec), and digital images were collected with a Spot 4.0 camera (Diagnostic Instruments).

TABLE 2
List of primary antibodies used for
immunohistochemical assessments.

| Antibody | Dilution | Source |
|---|---|---|
| Mouse monoclonal anti-BEST1 | 1:400 | Ab2182; Abcam |
| Mouse monoclonal anti-EZRIN | 1:400 | Ab4069; Abcam |
| Mouse monoclonal anti-RPE65 | 1:500 | NB100-355; Novus |
| | | Biologicals |
| Mouse monoclonal anti-BEST1 | 1:1,000 | MAB5316; Millipore |
| Rabbit polyclonal anti-hCAR | 1:10,000 | Courtesy of C. M. |
| | | Craft, University of |
| | | Southern California, |
| | | Los Angeles |
| Rabbit polyclonal anti- | 1:100 | AB5405; Millipore |
| RED/GREEN OPSIN | | |
| Rabbit polyclonal anti- | 1:5,000 | AB5407; Millipore |
| BLUE OPSIN | | |
| Rabbit polyclonal anti- | 1:500 | Courtesy of N. J. |
| SLC16A1 | | Philp, Thomas Jefferson |
| | | University, Philadelphia |
| Chicken polyclonal anti- | 1:500 | Michigan State |
| CNGB3 | | University |

### Confocal Microscopy and Image Analysis.

Confocal images were acquired on a TCS-SP5 confocal microscope system (Leica Microsystems) or an A1R laser scanning confocal microscope (Nikon Instruments). To obtain counts of cone-associated MV (cone-MV), two adjacent fields, each region of interest (ROI) 155 μm long, were imaged 4 mm from the optic nerve head in 10 retinal sections per retinal quadrant (temporal, superior, inferior, and nasal) (n=80 ROIs per eye) in both eyes from 6-wkold cBest (R25*/P463fs) and an age-matched WT control. Image stacks were acquired at 0.25-μm Z-steps and deconvolved with Huygens deconvolution software version 17.04 (Scientific Volume Imaging). All deconvolved images were rendered in the Leica LAS X 3D rendering module, where the cone-MV were counted manually. The length of both cone- and rod-MV was assessed within the Leica LAS X software from maximum projection images. Data were analyzed in Microsoft Excel and quantified using Prism software version 7 (GraphPad).

### REFERENCES

1. Bramall A N, Wright A F, Jacobson S G, McInnes R R (2010) The genomic, biochemical, and cellular responses of the retina in inherited photoreceptor degenerations and prospects for the treatment of these disorders. Annu Rev Neurosci 33:441-472.
2. Veleri S, et al. (2015) Biology and therapy of inherited retinal degenerative disease: Insights from mouse models. Dis Model Mech 8:109-129.
3. Cideciyan A V (2010) Leber congenital amaurosis due to RPE65 mutations and its treatment with gene therapy. Prog Retin Eye Res 29:398-427.
4. Ghazi N G, et al. (2016) Treatment of retinitis pigmentosa due to MERTK mutations by ocular subretinal injection of adeno-associated virus gene vector: Results of a phase I trial. Hum Genet 135:327-343.
5. Russell S, et al. (2017) Efficacy and safety of voretigene neparvovec (AAV2-hRPE65v2) in patients with RPE65-mediated inherited retinal dystrophy: A randomised, controlled, open-label, phase 3 trial. Lancet 390:849-860.
6. Best F (1905) Uber eine hereditare Maculaaffektion. Z Augenheilkd 13:199-212.
7. Petrukhin K, et al. (1998) Identification of the gene responsible for Best macular dystrophy. Nat Genet 19:241-247.
8. Boon C J, et al. (2009) The spectrum of ocular phenotypes caused by mutations in the BEST1 gene. Prog Retin Eye Res 28:187-205.
9. Marmorstein A D, et al. (2000) Bestrophin, the product of the Best vitelliform macular dystrophy gene (VMD2), localizes to the basolateral plasma membrane of the retinal pigment epithelium. Proc Natl Acad Sci USA 97:12758-12763.
10. Sun H, Tsunenari T, Yau K W, Nathans J (2002) The vitelliform macular dystrophy protein defines a new family of chloride channels. Proc Natl Acad Sci USA 99: 4008-4013.
11. Neussert R, Muller C, Milenkovic V M, Strauss O (2010) The presence of bestrophin1 modulates the Ca2+ recruitment from Ca2+ stores in the ER. Pflugers Arch 460: 163-175.
12. Hartzell H C, Qu Z, Yu K, Xiao Q, Chien L T (2008) Molecular physiology of bestrophins: Multifunctional membrane proteins linked to Best disease and other retinopathies. Physiol Rev 88:639-672.
13. Strauß O, Muller C, Reichhart N, Tamm E R, Gomez N M (2014) The role of bestrophin1 in intracellular Ca(2+) signaling. Adv Exp Med Biol 801:113-119.
14. Fischmeister R, Hartzell H C (2005) Volume sensitivity of the bestrophin family of chloride channels. J Physiol 562:477-491.
15. Milenkovic A, et al. (2015) Bestrophin 1 is indispensable for volume regulation in human retinal pigment epithelium cells. Proc Natl Acad Sci USA 112:E2630-E2639.
16. Mullins R F, Kuehn M H, Faidley E A, Syed N A, Stone E M (2007) Differential macular and peripheral expression of bestrophin in human eyes and its implication for Best disease. Invest Ophthalmol Vis Sci 48:3372-3380.
17. Guziewicz K E, et al. (2017) Bestrophinopathy: An RPE-photoreceptor interface disease. Prog Retin Eye Res 58:70-88.
18. Kane Dickson V, Pedi L, Long S B (2014) Structure and insights into the function of a Ca(2+)-activated Cl(−) channel. Nature 516:213-218.
19. Yang T, et al. (2014) Structure and selectivity in bestrophin ion channels. Science 346: 355-359.
20. Vaisey G, Miller A N, Long S B (2016) Distinct regions that control ion selectivity and calcium-dependent activation in the bestrophin ion channel. Proc Natl Acad Sci USA 113:E7399-E7408.
21. Deutman A F (1969) Electro-oculography in families with vitelliform dystrophy of the fovea. Detection of the carrier state. Arch Ophthalmol 81:305-316.
22. Arden G B, Constable P A (2006) The electro-oculogram. Prog Retin Eye Res 25: 207-248.
23. Guziewicz K E, et al. (2007) Bestrophin gene mutations cause canine multifocal retinopathy: A novel animal model for best disease. Invest Ophthalmol Vis Sci 48: 1959-1967.
24. Beltran W A, et al. (2014) Canine retina has a primate fovea-like bouquet of cone photoreceptors which is affected by inherited macular degenerations. PLoS One 9: e90390.
25. Hageman G S, Johnson L V (1991) The photoreceptor-retinal pigment epithelium interface. Principles and Practise of Clinical Electrophysiology of Vision, eds Heckenlively J R, Arden G B (Mosby-Year Book, St. Louis), pp 53-68.
26. Strauss O (2005) The retinal pigment epithelium in visual function. Physiol Rev 85: 845-881.
27. Huang B, Karwoski C J (1992) Light-evoked expansion of subretinal space volume in the retina of the frog. J Neurosci 12:4243-4252.
28. Putting B J, Zweypfenning R C, Vrensen G F, Oosterhuis J A, van Best J A (1992) Bloodretinal barrier dysfunction at the pigment epithelium induced by blue light. Invest Ophthalmol Vis Sci 33:3385-3393.
29. Li J D, Govardovskii V I, Steinberg R H (1994) Light-dependent hydration of the space surrounding photoreceptors in the cat retina. Vis Neurosci 11:743-752.
30. Huang Y, et al. (1998) Relation of optical coherence tomography to microanatomy in normal and rd chickens. Invest Ophthalmol Vis Sci 39:2405-2416.
31. Pianta M J, et al. (2003) In vivo micropathology of Best macular dystrophy with optical coherence tomography. Exp Eye Res 76:203-211.
32. Cideciyan A V, et al. (2005) In vivo dynamics of retinal injury and repair in the rhodopsin mutant dog model of human retinitis pigmentosa. Proc Natl Acad Sci USA 102: 5233-5238.
33. Bizheva K, et al. (2006) Optophysiology: Depth-resolved probing of retinal physiology with functional ultrahigh-resolution optical coherence tomography. Proc Natl Acad Sci USA 103:5066-5071.
34. Kay C N, et al. (2012) Three-dimensional distribution of the vitelliform lesion, photoreceptors, and retinal pigment epithelium in the macula of patients with best vitelliform macular dystrophy. Arch Ophthalmol 130:357-364.
35. Bissig D, Berkowitz B A (2012) Light-dependent changes in outer retinal water diffusion in rats in vivo. Mol Vis 18:2561.
36. Abràmoff M D, et al. (2013) Human photoreceptor outer segments shorten during light adaptation. Invest Ophthalmol Vis Sci 54:3721-3728.
37. Li Y, Fariss R N, Qian J W, Cohen E D, Qian H (2016) Light-induced thickening of photoreceptor outer segment layer detected by ultra-high resolution OCT imaging. Invest Ophthalmol Vis Sci 57:OCT105-OCT111.
38. Lu Y, Wang B, Pepperberg D R, Yao X (2016) Stimulus-evoked outer segment changes occur before the hyperpolarization of retinal photoreceptors. Biomed Opt Express 8: 38-47.
39. Zhang P, et al. (2017) In vivo optophysiology reveals that G-protein activation triggers osmotic swelling and increased light scattering of rod photoreceptors. Proc Natl Acad Sci USA 114:E2937-E2946.
40. Lu C D, et al. (2017) Photoreceptor layer thickness changes during dark adaptation observed with ultrahigh-resolution optical coherence tomography. Invest Ophthalmol Vis Sci 58:4632-4643.
41. Steinberg R H (1985) Interactions between the retinal pigment epithelium and the neural retina. Doc Ophthalmol 60:327-346.
42. Mieziewska K (1996) The interphotoreceptor matrix, a space in sight. Microsc Res Tech 35:463-471.
43. Sidjanin D J, et al. (2002) Canine CNGB3 mutations establish cone degeneration as orthologous to the human achromatopsia locus ACHM3. Hum Mol Genet 11: 1823-1833.
44. Komáromy A M, et al. (2013) Transient photoreceptor deconstruction by CNTF enhances rAAV-mediated cone functional rescue in late stage CNGB3-achromatopsia. Mol Ther 21:1131-1141.
45. Acland G M, Aguirre G D (1987) Retinal degenerations in the dog: IV. Early retinal degeneration (erd) in Norwegian elkhounds. Exp Eye Res 44:491-521.
46. Guziewicz K E, et al. (2013) Recombinant AAV-mediated BEST1 transfer to the retinal pigment epithelium: Analysis of serotype-dependent retinal effects. PLoS One 8: e75666.
47. Bruewer A R, et al. (2013) Evaluation of lateral spread of transgene expression following subretinal AAV-mediated gene delivery in dogs. PLoS One 8:e60218.
48. Beltran W A, et al. (2017) Optimization of retinal gene therapy for X-linked retinitis pigmentosa due to RPGR mutations. Mol Ther 25:1866-1880.
49. Sparrow J R, Hicks D, Hamel C P (2010) The retinal pigment epithelium in health and disease. Curr Mol Med 10:802-823.
50. Jacobson S G, et al. (2008) Photoreceptor layer topography in children with Leber congenital amaurosis caused by RPE65 mutations. Invest Ophthalmol Vis Sci 49: 4573-4577.
51. Charbel Issa P, et al. (2009) Characterisation of severe rod-cone dystrophy in a consanguineous family with a splice site mutation in the MERTK gene. Br J Ophthalmol 93:920-925.
52. Meunier I, et al. (2016) A dominant mutation in MAPKAPK3, an actor of p38 signaling pathway, causes a new retinal dystrophy involving Bruch's membrane and retinal pigment epithelium. Hum Mol Genet 25:916-926.
53. Singh R, et al. (2013) iPS cell modeling of Best disease: Insights into the pathophysiology of an inherited macular degeneration. Hum Mol Genet 22:593-607.
54. Bonilha V L, Finnemann S C, Rodriguez-Boulan E (1999) Ezrin promotes morphogenesis of apical microvilli and basal infoldings in retinal pigment epithelium. J Cell Biol 147: 1533-1548.
55. Koltzscher M, Neumann C, König S, Gerke V (2003) Ca2+-dependent binding and activation of dormant Ezrin by dimeric S100P. Mol Biol Cell 14:2372-2384.
56. Bonilha V L, et al. (2006) The retinal pigment epithelium apical microvilli and retinal function. Adv Exp Med Biol 572:519-524.
57. Bonilha V L, Rayborn M E, Saotome I, McClatchey A I, Hollyfield J G (2006) Microvilli defects in retinas of ezrin knockout mice. Exp Eye Res 82:720-729.
58. Philp N J, Nachmias V T (1985) Components of the cytoskeleton in the retinal pigmented epithelium of the chick. J Cell Biol 101:358-362.
59. Bonilha V L, et al. (2004) Proteomic characterization of isolated retinal pigment epithelium microvilli. Mol Cell Proteomics 3:1119-1127.
60. Gao H, Hollyfield J G (1992) Aging of the human retina. Differential loss of neurons and retinal pigment epithelial cells. Invest Ophthalmol Vis Sci 33:1-17.
61. Kiser P D, Golczak M, Palczewski K (2014) Chemistry of the retinoid (visual) cycle. Chem Rev 114:194-232.
62. Miller S S, Steinberg R H (1977) Passive ionic properties of frog retinal pigment epithelium. J Membr Biol 36:337-372.
63. Volland S, Esteve-Rudd J, Hoo J, Yee C, Williams D S (2015) A comparison of some organizational characteristics of the mouse central retina and the human macula. PLoS One 10:e0125631.
64. Cuenca N, Ortuño-Lizarán I, Pinilla I (Oct. 13, 2017) Cellular characterization of optical coherence tomography and outer retinal bands using specific immunohistochemistry markers and clinical implications. Ophthalmology, 10.1016/j.ophtha.2017.09.016.
65. Uehara F, Matthes M T, Yasumura D, LaVail M M (1990) Light-evoked changes in the interphotoreceptor matrix. Science 248:1633-1636.
66. Jonnal R S, Kocaoglu O P, Wang Q, Lee S, Miller D T (2012) Phase-sensitive imaging of the outer retina using optical coherence tomography and adaptive optics. Biomed Opt Express 3:104-124.
67. Hillmann D, et al. (2016) In vivo optical imaging of physiological responses to photostimulation in human photoreceptors. Proc Natl Acad Sci USA 113:13138-13143.
68. Bialek S, Miller S S (1994) K+ and Cl− transport mechanisms in bovine pigment epithelium that could modulate subretinal space volume and composition. J Physiol 475: 401-417.
69. Lu R, Levy A M, Zhang Q, Pittler S J, Yao X (2013) Dynamic near-infrared imaging reveals transient phototropic change in retinal rod photoreceptors. J Biomed Opt 18: 106013.
70. Querques G, et al. (2011) The spectrum of subclinical Best vitelliform macular dystrophy in subjects with mutations in BEST1 gene. Invest Ophthalmol Vis Sci 52: 4678-4684.
71. Qian C X, et al. (2017) Optical coherence tomography examination of the retinal pigment epithelium in Best vitelliform macular dystrophy. Ophthalmology 124: 456-463.
72. Kay D B, et al. (2013) Outer retinal structure in best vitelliform macular dystrophy. JAMA Ophthalmol 131:1207-1215.
73. Arora R, et al. (2016) Unilateral BEST1-associated retinopathy. Am J Ophthalmol 169: 24-32.
74. Scoles D, et al. (2017) Photoreceptor inner segment morphology in Best vitelliform macular dystrophy. Retina 37:741-748.
75. Abràmoff M D, Mullins R F, Stone E M (2014) Outer segment length in different Best disease genotypes. JAMA Ophthalmol 132:1152-1153.
76. Faktorovich E G, Steinberg R H, Yasumura D, Matthes M T, LaVail M M (1990) Photoreceptor degeneration in inherited retinal dystrophy delayed by basic fibroblast growth factor. Nature 347:83-86.
77. Milam A H, Li Z Y, Fariss R N (1998) Histopathology of the human retina in retinitis pigmentosa. Prog Retin Eye Res 17:175-205.
78. Jacobson S G, et al. (2006) Remodeling of the human retina in choroideremia: Rab escort protein 1 (REP-1) mutations. Invest Ophthalmol Vis Sci 47:4113-4120.
79. Jacobson S G, Cideciyan A V (2010) Treatment possibilities for retinitis pigmentosa. N Engl J Med 363:1669-1671.
80. Cideciyan A V, et al. (2011) Cone photoreceptors are the main targets for gene therapy of NPHP5 (IQCB1) or NPHP6 (CEP290) blindness: Generation of an all-cone Nphp6 hypomorph mouse that mimics the human retinal ciliopathy. Hum Mol Genet 20:1411-1423.
81. Sadigh S, et al. (2013) Abnormal thickening as well as thinning of the photoreceptor layer in intermediate age-related macular degeneration. Invest Ophthalmol Vis Sci 54: 1603-1612.
82. Beltran W A, et al. (2015) Successful arrest of photoreceptor and vision loss expands the therapeutic window of retinal gene therapy to later stages of disease. Proc Natl Acad Sci USA 112:E5844-E5853.
83. Johnson A A, et al. (2017) Bestrophin 1 and retinal disease. Prog Retin Eye Res 58: 45-69.
84. Schatz P, Klar J, Andréasson S, Ponjavic V, Dahl N (2006) Variant phenotype of Best vitelliform macular dystrophy associated with compound heterozygous mutations in VMD2. Ophthalmic Genet 27:51-56.
85. Burgess R, et al. (2008) Biallelic mutation of BEST1 causes a distinct retinopathy in humans. Am J Hum Genet 82:19-31.
86. Kinnick T R, et al. (2011) Autosomal recessive vitelliform macular dystrophy in a large cohort of vitelliform macular dystrophy patients. Retina 31:581-595.
87. Bitner H, et al. (2011) A homozygous frameshift mutation in BEST1 causes the classical form of Best disease in an autosomal recessive mode. Invest Ophthalmol Vis Sci 52: 5332-5338.
88. Boon C J, et al. (2013) Autosomal recessive bestrophinopathy: Differential diagnosis and treatment options. Ophthalmology 120:809-820.
89. Lamb T D, Pugh E N, Jr (2004) Dark adaptation and the retinoid cycle of vision. Prog Retin Eye Res 23:307-380.
90. Wang J S, Kefalov V J (2011) The cone-specific visual cycle. Prog Retin Eye Res 30: 115-128.
91. Kaylor J J, et al. (2013) Identification of DES1 as a vitamin A isomerase in Muller glial cells of the retina. Nat Chem Biol 9:30-36.
92. Cideciyan A V, Pugh E N, Jr, Lamb T D, Huang Y, Jacobson S G (1997) Rod plateau during dark adaptation in Sorsby's fundus dystrophy and vitamin A deficiency. Invest Ophthalmol Vis Sci 38:1786-1794.
93. Cideciyan A V, et al. (2000) Rod and cone visual cycle consequences of a null mutation in the 11-cis-retinol dehydrogenase gene in man. Vis Neurosci 17:667-678.
94. Cideciyan A V, et al. (2013) Human cone visual pigment deletions spare sufficient photoreceptors to warrant gene therapy. Hum Gene Ther 24:993-1006.
95. Cideciyan A V, et al. (2007) Reduced-illuminance autofluorescence imaging in ABCA4-associated retinal degenerations. J Opt Soc Am A Opt Image Sci Vis 24:1457-1467.
96. National Research Council (2011) Guide for the Care and Use of Laboratory Animals (Natl Acad Press, Washington, D.C.), 8th Ed.
97. Komáromy A M, Varner S E, de Juan E, Acland G M, Aguirre G D (2006) Application of a new subretinal injection device in the dog. Cell Transplant 15:511-519.
98. Delori F, et al. (2011) Quantitative measurements of autofluorescence with the scanning laser ophthalmoscope. Invest Ophthalmol Vis Sci 52:9379-9390.
99. Beltran W A, Hammond P, Acland G M, Aguirre G D (2006) A frameshift mutation in RPGR exon ORF15 causes photoreceptor degeneration and inner retina remodeling in a model of X-linked retinitis pigmentosa. Invest Ophthalmol Vis Sci 47:1669-1681.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A short hairpin RNA (shRNA) comprising:
a) a sense strand comprising the nucleotide sequence CGUCAAAGCUUCACAGUGU (SEQ ID NO: 2) and an antisense strand comprising the nucleotide sequence ACACUGUGAAGCUUUGACG (SEQ ID NO: 3); and
b) a loop.

2. The shRNA of claim 1, wherein the loop comprises the nucleotide sequence UUCAAGAGA (SEQ ID NO: 7).

3. The shRNA of claim 1, wherein the shRNA comprises the nucleotide sequence CGUCAAAGCUUCACAGUGUUUCAAGAGAACACUGUGAAGCUUUGACG (SEQ ID NO: 1).

4. A vector encoding the shRNA of claim 1.

5. The vector of claim 4 further comprising a recombinant bestrophin (BEST1) coding sequence that does not contain a sequence targeted by the shRNA.

6. The vector of claim 5, wherein the recombinant BEST1 coding sequence is codon-optimized for expression in a human cell.

7. The vector of claim 5, wherein the recombinant BEST1 coding sequence comprises a nucleotide sequence that is at least 90% identical to the nucleotide sequence of SEQ ID NO: 9.

8. The vector of claim 7, wherein the recombinant BEST1 coding sequence comprises the nucleotide sequence of SEQ ID NO: 9.

9. A vector encoding an shRNA of claim 1 and a recombinant BEST1 sequence comprising a nucleotide sequence that is at least 90% identical to the nucleotide sequence of SEQ ID NO: 11.

10. The vector of claim 9, wherein the vector comprises the nucleotide sequence of SEQ ID NO: 11.

11. The vector of claim 4, wherein the vector is a plasmid or a viral vector.

12. (canceled)

13. The vector of claim 11, wherein the viral vector is a recombinant adeno-associated viral (rAAV) vector.

14. The vector of claim 13, wherein the rAAV vector is self-complementary.

15. A recombinant adeno-associated viral (rAAV) particle comprising the rAAV vector of claim 13.

16. The rAAV particle of claim 15, wherein the rAAV viral particle is an AAV serotype 2 (AAV2) viral particle.

17. A composition comprising the rAAV particle of claim 15 and a pharmaceutically acceptable carrier.

18. A method of modulating BEST1 expression in a subject, the method comprising administering to the subject the composition of claim 17.

19. A method of treating Best Disease in a subject, the method comprising administering to the subject the composition of claim 17.

20. The method of claim 19, wherein the subject is a human subject.

21-24. (canceled)

25. A method of treating an autosomal recessive bestrophinopathy (ARB) in a human subject, the method comprising administering to the subject the composition of claim 17.

26-31. (canceled)

32. An shRNA that comprises a nucleotide sequence that differs from the nucleotide sequence of SEQ ID NO: 1 (CGUCAAAGCUUCACAGUGUUUCAAGAGAACACUGUGAAGCUUUGACG) by 1 or 2 nucleotides.
